SEQUENCE LISTING (22 sequences)

SEQ ID NO: 1 - protein, 428 aa, Homo sapiens
MAVGASGLEGDKMAGAMPLQLLLLLILLGPGNSLQLWDTWADEAEKALGPLLARDRRQATEYEYLDYDFLPETEPPEMLRNSTDTTPLTGPGTPESTTVEPAARRSTGLDAGGAVTELTTELANMGNLSTDSAAMEIQTTQPAATEAQTTQPVPTEAQTTPLAATEAQTTRLTATEAQTTPLAATEAQTTPPAATEAQTTQPTGLEAQTTAPAAMEAQTTAPAAMEAQTTPPAAMEAQTTQTTAMEAQTTAPEATEAQTTQPTATEAQTTPLAAMEALSTEPSATEALSMEPTTKRGLFIPFSVSSVTHKGIPMAASNLSVNYPVGAPDHISVKQCLLAILILALVATIFFVCTVVLAVRLSRKGHMYPVRNYSPTEMVCISSLLPDGGEGPSATANGGLSKAKSPGLTPEPREDREGDDLTLHSFLP

SEQ ID NO: 2 - DNA, 1287 bp, Homo sapiens
atggcagtgggggccagtggtctagaaggagataagatggctggtgccatgcctctgcaactcctcctgttgctgatcctactgggccctggcaacagcttgcagctgtgggacacctgggcagatgaagccgagaaagccttgggtcccctgcttgcccgggaccggagacaggccaccgaatatgagtacctagattatgatttcctgccagaaacggagcctccagaaatgctgaggaacagcactgacaccactcctctgactgggcctggaacccctgagtctaccactgtggagcctgctgcaaggcgttctactggcctggatgcaggaggggcagtcacagagctgaccacggagctggccaacatggggaacctgtccacggattcagcagctatggagatacagaccactcaaccagcagccacggaggcacagaccactcaaccagtgcccacggaggcacagaccactccactggcagccacagaggcacagacaactcgactgacggccacggaggcacagaccactccactggcagccacagaggcacagaccactccaccagcagccacggaagcacagaccactcaacccacaggcctggaggcacagaccactgcaccagcagccatggaggcacagaccactgcaccagcagccatggaagcacagaccactccaccagcagccatggaggcacagaccactcaaaccacagccatggaggcacagaccactgcaccagaagccacggaggcacagaccactcaacccacagccacggaggcacagaccactccactggcagccatggaggccctgtccacagaacccagtgccacagaggccctgtccatggaacctactaccaaaagaggtctgttcatacccttttctgtgtcctctgttactcacaagggcattcccatggcagccagcaatttgtccgtcaactacccagtgggggccccagaccacatctctgtgaagcagtgcctgctggccatcctaatcttggcgctggtggccactatcttcttcgtgtgcactgtggtgctggcggtccgcctctcccgcaagggccacatgtaccccgtgcgtaattactcccccaccgagatggtctgcatctcatccctgttgcctgatgggggtgaggggccctctgccacagccaatgggggcctgtccaaggccaagagcccgggcctgacgccagagcccagggaggaccgtgagggggatgacctcaccctgcacagcttcctcccttag

SEQ ID NO: 3 - protein, 923 aa, Homo sapiens
MERGLPLLCAVLALVLAPAGAFRNDKCGDTIKIESPGYLTSPGYPHSYHPSEKCEWLIQAPDPYQRIMINFNPHFDLEDRDCKYDYVEVFDGENENGHFRGKFCGKIAPPPVVSSGPFLFIKFVSDYETHGAGFSIRYEIFKRGPECSQNYTTPSGVIKSPGFPEKYPNSLECTYIVFAPKMSEIILEFESFDLEPDSNPPGGMFCRYDRLEIWDGFPDVGPHIGRYCGQKTPGRIRSSSGILSMVFYTDSAIAKEGFSANYSVLQSSVSEDFKCMEALGMESGEIHSDQITASSQYSTNWSAERSRLNYPENGWTPGEDSYREWIQVDLGLLRFVTAVGTQGAISKETKKKYYVKTYKIDVSSNGEDWITIKEGNKPVLFQGNTNPTDVVVAVFPKPLITRFVRIKPATWETGISMRFEVYGCKITDYPCSGMLGMVSGLISDSQITSSNQGDRNWMPENIRLVTSRSGWALPPAPHSYINEWLQIDLGEEKIVRGIIIQGGKHRENKVFMRKFKIGYSNNGSDWKMIMDDSKRKAKSFEGNNNYDTPELRTFPALSTRFIRIYPERATHGGLGLRMELLGCEVEAPTAGPTTPNGNLVDECDDDQANCHSGTGDDFQLTGGTTVLATEKPTVIDSTIQSEFPTYGFNCEFGWGSHKTFCHWEHDNHVQLKWSVLTSKTGPIQDHTGDGNFIYSQADENQKGKVARLVSPVVYSQNSAHCMTFWYHMSGSHVGTLRVKLRYQKPEEYDQLVWMAIGHQGDHWKEGRVLLHKSLKLYQVIFEGEIGKGNLGGIAVDDISINNHISQEDCAKPADLDKKNPEIKIDETGSTPGYEGEGEGDKNISRKPGNVLKTLDPILITIIAMSALGVLLGAVCGVVLYCACWHNGMSERNLSALENYNFELVDGVKLKKDKLNTQSTYSEA

SEQ ID NO: 4 - DNA, 2772 bp, Homo sapiens
atggagagggggctgccgctcctctgcgccgtgctcgccctcgtcctcgccccggccggcgcttttcgcaacgataaatgtggcgatactataaaaattgaaagccccgggtaccttacatctcctggttatcctcattcttatcacccaagtgaaaaatgcgaatggctgattcaggctccggacccataccagagaattatgatcaacttcaaccctcacttcgatttggaggacagagactgcaagtatgactacgtggaagtcttcgatggagaaaatgaaaatggacattttaggggaaagttctgtggaaagatagcccctcctcctgttgtgtcttcagggccatttctttttatcaaatttgtctctgactacgaaacacatggtgcaggattttccatacgttatgaaattttcaagagaggtcctgaatgttcccagaactacacaacacctagtggagtgataaagtcccccggattccctgaaaaatatcccaacagccttgaatgcacttatattgtctttgcgccaaagatgtcagagattatcctggaatttgaaagctttgacctggagcctgactcaaatcctccaggggggatgttctgtcgctacgaccggctagaaatctgggatggattccctgatgttggccctcacattgggcgttactgtggacagaaaacaccaggtcgaatccgatcctcatcgggcattctctccatggttttttacaccgacagcgcgatagcaaaagaaggtttctcagcaaactacagtgtcttgcagagcagtgtctcagaagatttcaaatgtatggaagctctgggcatggaatcaggagaaattcattctgaccagatcacagcttcttcccagtatagcaccaactggtctgcagagcgctcccgcctgaactaccctgagaatgggtggactcccggagaggattcctaccgagagtggatacaggtagacttgggccttctgcgctttgtcacggctgtcgggacacagggcgccatttcaaaagaaaccaagaagaaatattatgtcaagacttacaagatcgacgttagctccaacggggaagactggatcaccataaaagaaggaaacaaacctgttctctttcagggaaacaccaaccccacagatgttgtggttgcagtattccccaaaccactgataactcgatttgtccgaatcaagcctgcaacttgggaaactggcatatctatgagatttgaagtatacggttgcaagataacagattatccttgctctggaatgttgggtatggtgtctggacttatttctgactcccagatcacatcatccaaccaaggggacagaaactggatgcctgaaaacatccgcctggtaaccagtcgctctggctgggcacttccacccgcacctcattcctacatcaatgagtggctccaaatagacctgggggaggagaagatcgtgaggggcatcatcattcagggtgggaagcaccgagagaacaaggtgttcatgaggaagttcaagatcgggtacagcaacaacggctcggactggaagatgatcatggatgacagcaaacgcaaggcgaagtcttttgagggcaacaacaactatgatacacctgagctgcggacttttccagctctctccacgcgattcatcaggatctaccccgagagagccactcatggcggactggggctcagaatggagctgctgggctgtgaagtggaagcccctacagctggaccgaccactcccaacgggaacttggtggatgaatgtgatgacgaccaggccaactgccacagtggaacaggtgatgacttccagctcacaggtggcaccactgtgctggccacagaaaagcccacggtcatagacagcaccatacaatcagagtttccaacatatggttttaactgtgaatttggctggggctctcacaagaccttctgccactgggaacatgacaatcacgtgcagctcaagtggagtgtgttgaccagcaagacgggacccattcaggatcacacaggagatggcaacttcatctattcccaagctgacgaaaatcagaagggcaaagtggctcgcctggtgagccctgtggtttattcccagaactctgcccactgcatgaccttctggtatcacatgtctgggtcccacgtcggcacactcagggtcaaactgcgctaccagaagccagaggagtacgatcagctggtctggatggccattggacaccaaggtgaccactggaaggaagggcgtgtcttgctccacaagtctctgaaactttatcaggtgattttcgagggcgaaatcggaaaaggaaaccttggtgggattgctgtggatgacattagtattaataaccacatttcacaagaagattgtgcaaaaccagcagacctggataaaaagaacccagaaattaaaattgatgaaacagggagcacgccaggatacgaaggtgaaggagaaggtgacaagaacatctccaggaagccaggcaatgtgttgaagaccttagaccccatcctcatcaccatcatagccatgagtgccctgggggtcctcctgggggctgtctgtggggtcgtgctgtactgtgcctgttggcataatgggatgtcagaaagaaacttgtctgccctggagaactataactttgaacttgtggatggtgtgaagttgaaaaaagacaaactgaatacacagagtacttattcggaggcatga

SEQ ID NO: 5 - protein, 1132 aa, Homo sapiens
MPRAPRCRAVRSLLRSHYREVLPLATFVRRLGPQGWRLVQRGDPAAFRALVAQCLVCVPWDARPPPAAPSFRQVSCLKELVARVLQRLCERGAKNVLAFGFALLDGARGGPPEAFTTSVRSYLPNTVTDALRGSGAWGLLLRRVGDDVLVHLLARCALFVLVAPSCAYQVCGPPLYQLGAATQARPPPHASGPRRRLGCERAWNHSVREAGVPLGLPAPGARRRGGSASRSLPLPKRPRRGAAPEPERTPVGQGSWAHPGRTRGPSDRGFCVVSPARPAEEATSLEGALSGTRHSHPSVGRQHHAGPPSTSRPPRPWDTPCPPVYAETKHFLYSSGDKEQLRPSFLLSSLRPSLTGARRLVETIFLGSRPWMPGTPRRLPRLPQRYWQMRPLFLELLGNHAQCPYGVLLKTHCPLRAAVTPAAGVCAREKPQGSVAAPEEEDTDPRRLVQLLRQHSSPWQVYGFVRACLRRLVPPGLWGSRHNERRFLRNTKKFISLGKHAKLSLQELTWKMSVRDCAWLRRSPGVGCVPAAEHRLREEILAKFLHWLMSVYVVELLRSFFYVTETTFQKNRLFFYRKSVWSKLQSIGIRQHLKRVQLRELSEAEVRQHREARPALLTSRLRFIPKPDGLRPIVNMDYVVGARTFRREKRAERLTSRVKALFSVLNYERARRPGLLGASVLGLDDIHRAWRTFVLRVRAQDPPPELYFVKVDVTGAYDTIPQDRLTEVIASIIKPQNTYCVRRYAVVQKAAHGHVRKAFKSHVSTLTDLQPYMRQFVAHLQETSPLRDAVVIEQSSSLNEASSGLFDVFLRFMCHHAVRIRGKSYVQCQGIPQGSILSTLLCSLCYGDMENKLFAGIRRDGLLLRLVDDFLLVTPHLTHAKTFLRTLVRGVPEYGCVVNLRKTVVNFPVEDEALGGTAFVQMPAHGLFPWCGLLLDTRTLEVQSDYSSYARTSIRASLTFNRGFKAGRNMRRKLFGVLRLKCHSLFLDLQVNSLQTVCTNIYKILLLQAYRFHACVLQLPFHQQVWKNPTFFLRVISDTASLCYSILKAKNAGMSLGAKGAAGPLPSEAVQWLCHQAFLLKLTRHRVTYVPLLGSLRTAQTQLSRKLPGTTLTALEAAANPALPSDFKTILD

SEQ ID NO: 6 - DNA, 3399 bp, Homo sapiens
atgccgcgcgctccccgctgccgagccgtgcgctccctgctgcgcagccactaccgcgaggtgctgccgctggccacgttcgtgcggcgcctggggccccagggctggcggctggtgcagcgcggggacccggcggctttccgcgcgctggtggcccagtgcctggtgtgcgtgccctgggacgcacggccgccccccgccgccccctccttccgccaggtgtcctgcctgaaggagctggtggcccgagtgctgcagaggctgtgcgagcgcggcgcgaagaacgtgctggccttcggcttcgcgctgctggacggggcccgcgggggcccccccgaggccttcaccaccagcgtgcgcagctacctgcccaacacggtgaccgacgcactgcgggggagcggggcgtgggggctgctgctgcgccgcgtgggcgacgacgtgctggttcacctgctggcacgctgcgcgctctttgtgctggtggctcccagctgcgcctaccaggtgtgcgggccgccgctgtaccagctcggcgctgccactcaggcccggcccccgccacacgctagtggaccccgaaggcgtctgggatgcgaacgggcctggaaccatagcgtcagggaggccggggtccccctgggcctgccagccccgggtgcgaggaggcgcgggggcagtgccagccgaagtctgccgttgcccaagaggcccaggcgtggcgctgcccctgagccggagcggacgcccgttgggcaggggtcctgggcccacccgggcaggacgcgtggaccgagtgaccgtggtttctgtgtggtgtcacctgccagacccgccgaagaagccacctctttggagggtgcgctctctggcacgcgccactcccacccatccgtgggccgccagcaccacgcgggccccccatccacatcgcggccaccacgtccctgggacacgccttgtcccccggtgtacgccgagaccaagcacttcctctactcctcaggcgacaaggagcagctgcggccctccttcctactcagctctctgaggcccagcctgactggcgctcggaggctcgtggagaccatctttctgggttccaggccctggatgccagggactccccgcaggttgccccgcctgccccagcgctactggcaaatgcggcccctgtttctggagctgcttgggaaccacgcgcagtgcccctacggggtgctcctcaagacgcactgcccgctgcgagctgcggtcaccccagcagccggtgtctgtgcccgggagaagccccagggctctgtggcggcccccgaggaggaggacacagacccccgtcgcctggtgcagctgctccgccagcacagcagcccctggcaggtgtacggcttcgtgcgggcctgcctgcgccggctggtgcccccaggcctctggggctccaggcacaacgaacgccgcttcctcaggaacaccaagaagttcatctccctggggaagcatgccaagctctcgctgcaggagctgacgtggaagatgagcgtgcgggactgcgcttggctgcgcaggagcccaggggttggctgtgttccggccgcagagcaccgtctgcgtgaggagatcctggccaagttcctgcactggctgatgagtgtgtacgtcgtcgagctgctcaggtctttcttttatgtcacggagaccacgtttcaaaagaacaggctctttttctaccggaagagtgtctggagcaagttgcaaagcattggaatcagacagcacttgaagagggtgcagctgcgggagctgtcggaagcagaggtcaggcagcatcgggaagccaggcccgccctgctgacgtccagactccgcttcatccccaagcctgacgggctgcggccgattgtgaacatggactacgtcgtgggagccagaacgttccgcagagaaaagagggccgagcgtctcacctcgagggtgaaggcactgttcagcgtgctcaactacgagcgggcgcggcgccccggcctcctgggcgcctctgtgctgggcctggacgatatccacagggcctggcgcaccttcgtgctgcgtgtgcgggcccaggacccgccgcctgagctgtactttgtcaaggtggatgtgacgggcgcgtacgacaccatcccccaggacaggctcacggaggtcatcgccagcatcatcaaaccccagaacacgtactgcgtgcgtcggtatgccgtggtccagaaggccgcccatgggcacgtccgcaaggccttcaagagccacgtctctaccttgacagacctccagccgtacatgcgacagttcgtggctcacctgcaggagaccagcccgctgagggatgccgtcgtcatcgagcagagctcctccctgaatgaggccagcagtggcctcttcgacgtcttcctacgcttcatgtgccaccacgccgtgcgcatcaggggcaagtcctacgtccagtgccaggggatcccgcagggctccatcctctccacgctgctctgcagcctgtgctacggcgacatggagaacaagctgtttgcggggattcggcgggacgggctgctcctgcgtttggtggatgatttcttgttggtgacacctcacctcacccacgcgaaaaccttcctcaggaccctggtccgaggtgtccctgagtatggctgcgtggtgaacttgcggaagacagtggtgaacttccctgtagaagacgaggccctgggtggcacggcttttgttcagatgccggcccacggcctattcccctggtgcggcctgctgctggatacccggaccctggaggtgcagagcgactactccagctatgcccggacctccatcagagccagtctcaccttcaaccgcggcttcaaggctgggaggaacatgcgtcgcaaactctttggggtcttgcggctgaagtgtcacagcctgtttctggatttgcaggtgaacagcctccagacggtgtgcaccaacatctacaagatcctcctgctgcaggcgtacaggtttcacgcatgtgtgctgcagctcccatttcatcagcaagtttggaagaaccccacatttttcctgcgcgtcatctctgacacggcctccctctgctactccatcctgaaagccaagaacgcagggatgtcgctgggggccaagggcgccgccggccctctgccctccgaggccgtgcagtggctgtgccaccaagcattcctgctcaagctgactcgacaccgtgtcacctacgtgccactcctggggtcactcaggacagcccagacgcagctgagtcggaagctcccggggacgacgctgactgccctggaggccgcagccaacccggcactgccctcagacttcaagaccatcctggactga

SEQ ID NO: 7 - protein, 7 aa, Artificial Sequence
  Nuclear localization sequence
PKKKRKV

SEQ ID NO: 8 - protein, 16 aa, Artificial Sequence
  Nuclear localization sequence
KRPAATKKAGQAKKKK

SEQ ID NO: 9 - protein, 13 aa, Artificial Sequence
  Protein transduction domain (PTD)
GRKKRRQRRRPPQ

SEQ ID NO: 10 - protein, 18 aa, Artificial Sequence
  Protein transduction domain
LLIILRRRIRKQAHAHSK

SEQ ID NO: 11 - protein, 27 aa, Artificial Sequence
  Protein transduction domain
GWTLNSAGYLLGKINLKALAALAKKIL

SEQ ID NO: 12 - protein, 27 aa, Artificial Sequence
  Protein transduction domain
GALFLGFLGAAGSTMGAWSQPKKKRKV

SEQ ID NO: 13 - protein, 18 aa, Artificial Sequence
  Protein transduction domain
KLALKLALKALKAALKLA

SEQ ID NO: 14 - protein, 9 aa, Artificial Sequence
  Protein transduction domain
RRWWRRWRR

SEQ ID NO: 15 - protein, 9 aa, Artificial Sequence
  Protein transduction domain
RRRRRRRRR

SEQ ID NO: 16 - protein, 16 aa, Artificial Sequence
  Protein tranduction domain
RQIKIWFQNRRMKWKK

SEQ ID NO: 17 - protein, 33 aa, Artificial Sequence
  Protein transduction domain
DAATATRGRSAASRPTERPRAPARSASRPRRVD

SEQ ID NO: 18 - protein, 12 aa, Artificial Sequence
  Protein transduction domain
AAVLLPVLLAAP

SEQ ID NO: 19 - protein, 26 aa, Artificial Sequence
  Protein transduction domain
DPKGDPKGVTVTVTVTVTGKGDPKPD

SEQ ID NO: 20 - protein, 18 aa, Artificial Sequence
  Protein transduction domain
RGGRLSYSRRRFSTSTGR

SEQ ID NO: 21 - protein, 21 aa, Artificial Sequence
  T2A self-cleaving peptide
GSGEGRGSLLTCGDVEENPGP

SEQ ID NO: 22 - protein, 10 aa, Artificial Sequence
  ligand-dependent destabilization domain
MGVQVETISP

(19) United States
(12) Patent Application Publication
(10) Pub. No.: US 2020/0281986 A1
(43) Pub. Date: Sep. 10, 2020

(54) ENGINEERING MESODERMAL PRECURSOR CELL COMPOSITIONS FOR THE TREATMENT OR PROPHYLAXIS OF PERFUSION DISORDERS

(71) Applicant: VASCUGEN, INC., Indianapolis, IN (US)

(72) Inventor: Mervin C. Yoder, Indianapolis, IN (US)

(73) Assignee: VASCUGEN, INC., Indianapolis, IN (US)

(21) Appl. No.: 16/562,958

(22) Filed: Sep. 6, 2019

Related U.S. Application Data
(60) Provisional application No. 62/728,191, filed on Sep. 7, 2018.

(51) Int. Cl. A61K 35/36; C12N 5/071; C12N 15/85; A61P 17/02

(52) U.S. Cl. CPC A61K 35/36; C12N 5/0625; C12N 2506/45; A61P 17/02; C12N 2510/00; C12N 15/85

(57) ABSTRACT

The present disclosure describes the engineering of a mesodermal precursor cell population obtained through the differentiation of induced pluripotent stem cells (iPSCs) for the cell therapy treatment of perfusion disorders. The modifications improve the survival and clonal proliferation of the mesodermal precursor cell population ex vivo and facilitate their migration to sites of ischemic injury in vivo.

### CROSS-REFERENCE TO RELATED APPLICATIONS

This application claims the benefit of U.S. Provisional Patent Application 62/728,191, filed Sep. 7, 2018, the content of which is incorporated by reference herein in its entirety.

### STATEMENT REGARDING FEDERALLY SPONSORED RESEARCH

This invention was made with government support under DK063114 awarded by National Institutes of Health. The government has certain rights in the invention.

### REFERENCE TO SEQUENCE LISTING SUBMITTED ELECTRONICALLY VIA EFS-WEB

The content of the electronically submitted sequence listing (Name: 3094VG01UTL1US_sequence_listing_ST25.txt, Size: 36,189 bytes; and Date of Creation: Jan. 3, 2019) is incorporated herein by reference in its entirety.

### FIELD OF THE DISCLOSURE

The present disclosure pertains generally to the engineering of mesodermal precursor cell compositions and their use for the treatment or prophylaxis of perfusion disorders.

### BACKGROUND OF THE DISCLOSURE

Perfusion disorders compromise the delivery of oxygenated blood to tissues, organs and/or extremities as a result of physical trauma, systemic disease or vascular disease. In severe cases, the subsequent ischemia-reperfusion (FR) injury impairs vascular function resulting in parenchymal cell damage and tissue/organ injury. The ensuing tissue damage is permanent and often leads to life-changing disability or even death. It is therefore not surprising that cardiovascular disease remains the leading cause of mortality in the world, more than cancer, chronic respiratory diseases, or accidents combined. According to the Center for Disease Control and Prevention, in the U.S. alone, an estimated 610,000 people die of heart disease every year.

Treatment of perfusion disorders requires rapid intervention to mitigate endothelial injury and reestablish vital oxygenation of surrounding tissues. Laser and balloon angioplasty in combination with drug eluting stents are the most common ways of treating occluded arteries without open surgery. These procedures are not however without risk to the patient either from arterial dissection, arterial perforation, subsequent restenosis or thrombosis.

Over the past two decades, cellular therapies have been investigated for the potential treatment of ischemia. In mammals, blood vessels can form either by angiogenesis or vasculogenesis. Angiogenesis is a postnatal process by which blood vessels are formed from existing vasculature (e.g. sprouting angiogenesis). In contrast, vasculogenesis refers to the de novo formation of blood vessels initiated by circulating endothelial progenitor cells. Once thought to be restricted solely to embryogenesis, postnatal vasculogenesis is now recognized to be a key mechanism by which neovascularization is induced at sites of ischemic injury (Asahara et al. Circulation (1995) 91 2793-801). For example, preliminary studies indicate human endothelial colony forming cells (ECFCs) can enhance vascular repair and improve blood flow following myocardial infarction (Dubois et al. J Am Coll Cardiol (2010) 55, 2232-2243; Schuh et al. Basic Res Cardiol (2008) 103, 69-77, stroke (Moubarik et al. (2011) Stem Cell Rev 7, 208-220), ischemic retinopathy (Stitt et al. Prog Retin Eye Res (2001) 30, 149-166; Medina et al. Invest Ophthalmol Vis Sci (2010) 51, 5906-5913), ischemic limb injury (Schwartz et al. Arterioscler Thromb Vasc Biol 32, e13-21 (2012); Saif et al. (2010) Arterioscler Thromb Vasc Biol 30, 1897-1904; Bouvard et al. Arterioscler Thromb Vasc Biol (2010) 30, 1569-1575; Lee et al. Stem Cells 31, 666-681 (2013), as well as facilitate the engraftment and re-endothelialization of denuded vascular segments or implanted grafts (Stroncek et al. (2012) Acta Biomater 8, 201-208).

Although human endothelial colony forming cells (ECFCs) with clonal proliferative potential and intrinsic in vivo vessel forming ability have now been identified by several research groups, to date no unique markers have been identified to facilitate their isolation from circulating blood or determine their site of origin in adult humans (Yoder M C. Pulmonary Circulation. (2018) 8(1):2045893217743950). Indeed, efforts to find a viable approach to a cellular therapy for the treatment of perfusion disorders have been thwarted by the apparent lack of concordance amongst the reported endothelial cell populations broadly described in the literature as “endothelial progenitor cells” (Medina et al. Stem Cells Translational Medicine (2017) 6:1316-1320).

Thus, there remains a need in the art for a clonal endothelial precursor cell population having defined, uniform characteristics as well as the requisite proliferation potential needed for the cell therapy treatment of perfusion disorders.

### SUMMARY OF THE DISCLOSURE

The present disclosure describes engineered mesodermal precursor cell (MSD) compositions and methods for use in the treatment of various perfusion disorders, including ischemic and/or reperfusion injury to organs, tissues or extremities. In their native state, mesodermal precursor cells differentiate into cells of the endothelial cell lineage, but with the caveat that they can often exhibit low proliferative potential in culture that limits their survival in culture to a mere two or three days. The disclosed methods remedy these deficiencies by enhancing the clonal proliferation of MSD cell populations as well as their neovascularization potential in vivo. The present disclosure also describes engineered ECFC compositions and methods for use in the treatment of various perfusion disorders, including ischemic and/or reperfusion injury to organs, tissues or extremities. This approach also provides a vehicle for the targeted delivery of therapeutic compounds to sites of ischemic injury in vivo.

In a first aspect, an isolated population of engineered mesodermal precursor cells expressing at least one of KDR, NCAM and APLNR is disclosed wherein the precursor cells are engineered to enhance the non-neoplastic proliferation and survival of the mesodermal precursor cells and/or the migration of the mesodermal precursor cells and their progeny toward ischemic tissue.

In certain embodiments of the first aspect, the isolated population of engineered mesodermal precursor cells express at least two of KDR, NCAM and APLNR.

In certain embodiments of the first aspect, the isolated population of engineered mesodermal precursor cells express all three of KDR, NCAM and APLNR.

In certain embodiments of the first aspect, the mesodermal precursor cell population can differentiate into endothelial progenitor cells such as endothelial colony forming-like cells (ECFCs-like).

In certain embodiments of the first aspect, the mesodermal precursor cells can be engineered by gene editing.

In certain embodiments of the first aspect, the mesodermal precursor cells comprise an agent that enhances the non-neoplastic proliferation and/or survival of the mesodermal precursor cells and/or the migration of the mesodermal precursor cells and their progeny toward ischemic tissue.

In certain embodiments of the first aspect, the agent comprises a transgene and/or an mRNA.

In certain embodiments of the first aspect, the agent comprises a transgene or an mRNA encoding P selectin ligand 1 (PSGL-1), neuropilin-1 and/or telomerase or any portion thereof.

In certain embodiments of the first aspect, the transgene or mRNA encoding P selectin ligand 1 (PSGL-1) comprises a nucleotide sequence having at least 25 nucleotides of SEQ ID NO: 2, wherein the expression of the transgene or mRNA enhances the migration of the mesodermal precursor cells and their progeny toward ischemic tissue in vivo.

In certain embodiments of the first aspect, the transgene or mRNA encoding neuropilin-1 comprises a nucleotide sequence having at least 25 nucleotides of SEQ ID NO: 4, wherein the expression of the transgene or mRNA enhances the migration of the mesodermal precursor cells and their progeny toward ischemic tissue in vivo.

In certain embodiments of the first aspect, the transgene or mRNA encoding telomerase comprises a nucleotide sequence having at least 25 nucleotides of SEQ ID NO: 5, wherein the expression of the transgene or mRNA enhances the non-neoplastic proliferation and/or survival of the mesodermal precursor cells.

In certain embodiments of the first aspect, the agent comprises a transducible protein.

In certain embodiments of the first aspect, the transducible protein may comprise P selectin ligand 1 (PSGL-1), neuropilin-1 and/or telomerase or any portion thereof.

In certain embodiments of the first aspect, the expression of the transgene can be inducible.

In certain embodiments of the first aspect, the transgene can be episomal, chromosomally integrated, for example, at a genomic safe harbor site.

In certain embodiments of the first aspect, the transgene can be operably linked to a promoter of an endogenous gene that is expressed in the mesodermal precursor cell population.

In certain embodiments of the first aspect, the transgene can be placed downstream of an internal ribosomal entry site (IBES) and inserted into the 3′ untranslated region of an endogenous gene that is expressed in the mesodermal precursor cell population.

The endogenous gene can have a nucleotide sequence comprising at least 25 nucleotides of SEQ ID NO: 2 or SEQ ID NO: 4.

In certain embodiments of the first aspect, the mesodermal precursor cells can be derived from pluripotent stem cells expressing at least one stem cell transcription factor selected from the group consisting of NANOG, SOX2 and OCT4A.

In certain embodiments of the first aspect, the pluripotent stem cells are induced pluripotent stem cells (iPSCs).

In certain embodiments of the first aspect, the pluripotent stem cells are multipotent stem cells such as cord stem cells.

In certain embodiments of the first aspect, the pluripotent stem cells are embryonic stem cells, adult stem cells or induced pluripotent stem cells, e.g. induced pluripotent stem cells generated from the subject's somatic cells.

In a second aspect, a cell composition is disclosed comprising a first cell population of engineered mesodermal precursor cells and a second non-recombinant cell population comprising, for example, non-recombinant mesodermal precursor cells.

In a third aspect, a method for treating a perfusion disorder in a subject's organ, tissue and/or extremity is disclosed comprising administering a cellular composition comprising a therapeutically effective amount of any one of the engineered mesodermal precursor cells disclosed herein.

In certain embodiments of the third aspect, the subject's organ, tissue and/or extremity can be irradiated prior to the administration of the cellular composition.

In certain embodiments of the third aspect, the subject's perfusion disorder can be caused by physical trauma to the subject's organ, tissue and/or extremity.

In certain embodiments of the third aspect, the subject's perfusion disorder can be a vascular disorder that, for example, causes an ischemia and/or reperfusion injury to the subject's organ, tissue and/or extremity.

In certain embodiments of the third aspect, the vascular disorder can be peripheral arterial disease (PAD) or critical limb ischemia (CLI). In certain embodiments of the third aspect, the subject's organ or tissue can be from the musculoskeletal system, circulatory system, nervous system, integumentary system, digestive system, respiratory system, immune system, urinary system, reproductive system or endocrine system. For example, the organ can be the subject's heart, lung, brain, liver or kidney and the tissue can be an epithelial, connective, muscular, or nervous tissue. In other examples, the tissue can be cerebral, myocardial, lung, renal, liver, skeletal, or peripheral tissue.

In certain embodiments of the third aspect, the administration of the cellular composition can (1) enhance blood flow through the subject's organ, tissue and/or extremity, (2) restore endothelial cell function in the subject's organ, tissue and/or extremity and/or (3) promote neovascularization in the subject's organ, tissue and/or extremity.

In certain embodiments of the third aspect, the cellular composition can be administered directly to the subject's organ, tissue and/or extremity in vivo.

In certain embodiments of the third aspect, the cellular composition can be administered directly to the subject's organ and/or tissue ex vivo prior its transplantation into the subject.

In certain embodiments of the third aspect, the cellular composition can be administered intravenously to the subject.

In certain embodiments of the third aspect, the subject has atherosclerosis, diabetes and/or cancer.

In a fourth aspect, an isolated population of engineered endothelial colony-forming cells (ECFCs) is disclosed wherein the ECFCs are engineered to enhance the non-neoplastic proliferation and survival of the cells and/or the migration of the cells and their progeny toward ischemic tissue.

In certain embodiments, the isolated population of ECFC-like cells express at least one marker chosen from CD31, NRP-1, CD144 and KDR.

In certain embodiments, the isolated population of ECFC-like cells express at least two markers chosen from CD31, NRP-1, CD144 and KDR.

In certain embodiments, the isolated population of ECFC-like cells express at least three markers chosen from CD31, NRP-1, CD144 and KDR.

In certain embodiments, the isolated population of ECFC-like cells express at least four markers chosen from CD31, NRP-1, CD144 and KDR.

In certain embodiments of the fourth aspect, the endothelial colony-forming cells (ECFCs) are high proliferative potential ECFCs ((HPP)-ECFCs).

In an embodiment of the fourth aspect, the endothelial colony-forming cells (ECFCs) do not express α-smooth muscle actin (α-SMA).

In certain embodiments of the fourth aspect, the ECFCs can be engineered by gene editing.

In certain embodiments of the fourth aspect, the ECFCs comprise an agent that enhances the non-neoplastic proliferation and/or survival of the cells and/or the migration of the cells and their progeny toward ischemic tissue.

In certain embodiments of the fourth aspect, the agent comprises a transgene and/or an mRNA.

In certain embodiments of the fourth aspect, the agent comprises a transgene or an mRNA encoding P selectin ligand 1 (PSGL-1), neuropilin-1 and/or telomerase or any portion thereof.

In certain embodiments of the fourth aspect, the transgene or mRNA encoding P selectin ligand 1 (PSGL-1) comprises a nucleotide sequence having at least 25 nucleotides of SEQ ID NO: 2, wherein the expression of the transgene or mRNA enhances the migration of the cells and their progeny toward ischemic tissue in vivo.

In certain embodiments of the fourth aspect, the transgene or mRNA encoding neuropilin-1 comprises a nucleotide sequence having at least 25 nucleotides of SEQ ID NO: 4, wherein the expression of the transgene or mRNA enhances the migration of the cells and their progeny toward ischemic tissue in vivo.

In certain embodiments of the fourth aspect, the transgene or mRNA encoding telomerase comprises a nucleotide sequence having at least 25 nucleotides of SEQ ID NO: 5, wherein the expression of the transgene or mRNA enhances the non-neoplastic proliferation and/or survival of the cells.

In certain embodiments of the fourth aspect, the agent comprises a transducible protein.

In certain embodiments of the fourth aspect, the transducible protein may comprise P selectin ligand 1 (PSGL-1), neuropilin-1 and/or telomerase or any portion thereof.

In certain embodiments of the fourth aspect, the expression of the transgene can be inducible.

In certain embodiments of the fourth aspect, the transgene can be episomal, chromosomally integrated, for example, at a genomic safe harbor site.

In certain embodiments of the fourth aspect, the transgene can be operably linked to a promoter of an endogenous gene that is expressed in the ECFC population.

In certain embodiments of the fourth aspect, the transgene can be placed downstream of an internal ribosomal entry site (IRES) and inserted into the 3′ untranslated region of an endogenous gene that is expressed in the ECFC population.

The endogenous gene can have a nucleotide sequence comprising at least 25 nucleotides of SEQ ID NO: 2 or SEQ ID NO: 4.

In certain embodiments of the fourth aspect, the ECFCs can be derived from pluripotent stem cells expressing at least one stem cell transcription factor selected from the group consisting of NANOG, SOX2 and OCT4A.

In certain embodiments of the fourth aspect, the pluripotent stem cells are induced pluripotent stem cells (iPSCs).

In certain embodiments of the fourth aspect, the endothelial colony-forming cells (ECFCs) are derived from multipotent stem cells such as cord stem cells.

In certain embodiments of the fourth aspect, the endothelial colony-forming cells (ECFCs) are derived from pluripotent stem cells.

In certain embodiments of the fourth aspect, the pluripotent stem cells express at least one of the transcription factors selected from the group consisting of OCT4A, NANOG, and SOX2.

In certain embodiments of the fourth aspect, endothelial colony-forming cells (ECFCs) are derived from pluripotent stem cells without co-culture with bone marrow cells.

In certain embodiments of the fourth aspect, the endothelial colony-forming cells (ECFCs) are derived from pluripotent stem cells without embryoid body formation.

### BRIEF DESCRIPTION OF THE DRAWINGS

These and other features of the disclosure will become more apparent in the following detailed description in which reference is made to the appended drawings wherein:

FIG. 1, presented as FIGS. 1A-1E, shows the CRISPR/cas targeting of the 3′ untranslated region (UTR) of the endogenous PSGL-1 gene in human iPSCs using the HR180PA-1 targeting vector and the pX330 hSpCas9 expression vector.

FIG. 1A depicts an example of the different steps required for generating a PSGL1 Gene Knock-in in induced pluripotent stem cells (iPSCs). The flowchart shows a schematic of the PSGL1 genomic sequence with the CRISPR/cas9 target site in the 3′untranslated region of PSGL1, an IRES-GFP co-expression homologous recombination HR180PA-1 targeting vector (IRES-GFP-pA-loxP-MCS1-EF1a-RFP-T2A-Puro-pA-LoxP-MCS2) containing homology arms that target PSGL1 gene sequences and a pX330-U6-Chimeric_BB-CBh-hSpCas9 expression vector in which a U6 snRNA promoter and a CBh promoter (a hybrid between the cytomegalovirus (CMV) and chicken □-actin (CBA)) drive the expression of a PSGL1-specific sgRNA sequence and human codon-optimized S. pyogenes Cas9 (SpCas9) respectively. IRES: internal ribosomal entry site; GFP: green fluorescent protein; RFP: tdTomato red fluorescent protein.

FIG. 1B depicts an exemplary FACS analysis of iPSCs containing the PSGL1 Gene Knock-in. No green fluorescence was detected because the PSGL1 promoter is not active in iPSCs. The presence of tdTomato red fluorescence confirms the HR180PA-1 targeting vector was successfully knocked-in to the PSGL1 locus.

FIG. 1C shows an exemplary depiction of the HR180PA-1 expression vector.

FIG. 1D shows an exemplary depiction of the pX330 hSpCas9 expression vector.

FIG. 1E shows an exemplary depiction of an inducible hTERT expression vector.

FIG. 2, presented as FIGS. 2A-2B, shows an exemplary cre-lox mediated recombination within the targeting vector that has been knocked into the 3′ untranslated region (UTR) of the endogenous PSGL-1 gene of human iPSCs.

FIG. 2A depicts an exemplary schematic of the Cre recombinase excision between the two loxP sites in the HR180PA-1 targeting vector (IRES-GFP-pA-loxP-MCS1-EF1a-RFP-T2A-Puro-pA-LoxP-MCS2). FIG. 2B depicts an exemplary FACS analysis of iPSCs containing the PSGL1 Gene Knock-in after Cre recombinase excision which results in the appearance of iPSCs having no red fluorescence.

FIG. 3, presented as FIGS. 3A-3B, shows the detection of green fluorescence in PSGL-1 targeting iPSCs after endothelial differentiation.

FIG. 3A depicts an exemplary FACS analysis of iPSCs containing the PSGL1 Gene Knock-in after differentiation toward the endothelial lineage. The differentiation is accompanied by the appearance of iPSCs having green fluorescence.

FIG. 3B shows the appearance of green fluorescence in cells containing the PSGL1 Gene Knock-in after differentiation of the cells into colony-forming unit-granulocyte, erythroid, macrophage, megakaryocyte (CFU-GEMM) colonies. CFU-GEMM colonies include myeloid cells that express PSGL1 (Laszik et al., Blood, (1996) 88, No. 8, 3010-3021).

FIG. 4, presented as FIGS. 4A-4B, depicts the TALEN-mediated dual GFP/tdTomato reporters used to target both alleles of the endogenous CLYBL locus that resulted in GFP/tdTomato fluorescence in iPSC colonies.

FIG. 4A depicts an exemplary method of transcription activator-like effector nuclease (TALEN)-mediated dual GFP/tdTomato reporter targeting of both alleles of the endogenous CLYBL locus in human induced pluripotent stem cell (iPSC).

FIG. 4B shows GFP/tdTomato expression in iPSC colonies having the GFP/tdTomato reporter transgenes knocked-in to both alleles of the endogenous CLYBL locus.

FIG. 5 depicts an exemplary method of transcription activator-like effector nuclease (TALEN)-mediated targeting of the EF1a-FKBP12-hTert-IRES-RFP-Neo donor vector to one of the alleles of the endogenous CLYBL safe harbor locus in human induced pluripotent stem cell (iPSC).

FIG. 6 depicts an exemplary method of transcription activator-like effector nuclease (TALEN)-mediated targeting of the EF1a-FKBP12-PSGL-IRES-RFP-Neo or EF1a-FKBP12-NEUROPILIN-IRES-RFP-Neo donor vector to one of the alleles of the endogenous CLYBL safe harbor locus in human induced pluripotent stem cell (iPSC).

Other features and advantages of the disclosure will be apparent from the following Detailed Description and from the Exemplary Embodiments.

### DETAILED DESCRIPTION OF THE DISCLOSURE

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described herein. The materials, methods, and examples are illustrative only and not intended to be limiting. Other features of the disclosure are apparent from the following detailed description and the claims.

### Definitions

As used herein, the singular forms “a,” “an,” and “the,” are intended to include the plural forms as well, unless the context clearly indicates otherwise.

The phrase “and/or,” as used herein in the specification and in the claims, should be understood to mean “either or both” of the elements so conjoined, i.e., elements that are conjunctively present in some cases and disjunctively present in other cases. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

When the term “about” is used in conjunction with a numerical range, it modifies that range by extending the boundaries above and below those numerical values. In general, the term “about” is used herein to modify a numerical value above and below the stated value by a variance of 20%, 10%, 5%, or 1%. In certain embodiments, the term “about” is used to modify a numerical value above and below the stated value by a variance of 10%. In certain embodiments, the term “about” is used to modify a numerical value above and below the stated value by a variance of 5%. In certain embodiments, the term “about” is used to modify a numerical value above and below the stated value by a variance of 1%.

When a range of values is listed herein, it is intended to encompass each value and sub-range within that range. For example, “1-5 ng” is intended to encompass 1 ng, 2 ng, 3 ng, 4 ng, 5 ng, 1-2 ng, 1-3 ng, 1-4 ng, 1-5 ng, 2-3 ng, 2-4 ng, 2-5 ng, 3-4 ng, 3-5 ng, and 4-5 ng.

It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

As used herein, the term “isolated” refers to a stem cell or population of daughter stem cells in a non-naturally occurring state outside of the body (e.g., isolated from the body or a biological sample from the body).

By a “population of cells” or “cell population” is meant a collection of at least ten cells. Preferably, the population consists of at least twenty cells, more preferably at least one hundred cells, and most preferably at least one thousand, or even one million cells. Because the stem cells of the present invention exhibit a capacity for self-renewal, they can be expanded in culture to produce populations of billions of cells.

A “subject” is a vertebrate, preferably a mammal (e.g., a non-human mammal), more preferably a primate and still more preferably a human. Mammals include, but are not limited to, primates, humans, farm animals, rodents, sport animals, and pets.

As used herein, the term “agent” is meant to encompass any molecule, chemical entity, composition, drug, therapeutic agent, or biological agent capable of enhancing the non-neoplastic proliferation and survival of the mesodermal precursor cells and/or the migration of the mesodermal precursor cells and their progeny toward ischemic tissue. The term includes, but is not limited to, nucleic acids (such as transgenes, mRNAs, non-coding RNAs), proteins, small molecules or enzyme inhibitors (e.g. kinase or histone deacetylase inhibitors etc.).

As used herein, the term “expression” includes transcription and translation.

As used herein, the term “gene” refers to a DNA sequence that encodes through its template or messenger RNA a sequence of amino acids characteristic of a specific peptide, polypeptide, or protein. The term “gene” also refers to a DNA sequence that encodes a non-coding RNA product. The term gene as used herein with reference to genomic DNA includes intervening, non-coding regions as well as regulatory regions and can include 5′ and 3′ ends.

As used herein, the term “vector” refers to a replicon, such as a plasmid, phage, or cosmid, into which another DNA segment may be inserted so as to bring about the replication of the inserted segment. The vectors can be expression vectors.

As used herein, the term “expression vector” refers to a vector that includes one or more transcription regulatory sequences.

As used herein, the term “transcription regulatory sequence” refers to a DNA sequence that controls and regulates the transcription and/or translation of another DNA sequence. In eukaryotes, transcription regulatory sequences include, but are not limited to, promoters, enhancers, polyadenylation signals and silencers.

As used herein, the terms “transformed,” “transgenic,” “transfected” and “recombinant” refer to a host organism into which a heterologous nucleic acid molecule has been introduced. The nucleic acid molecule can be stably integrated into the genome of the host or the nucleic acid molecule can also be present as an extrachromosomal molecule. Such an extrachromosomal molecule can be auto-replicating. Transformed cells are understood to encompass not only the end product of a transformation process, but also transgenic progeny thereof. A “non-transformed,” “non-transgenic,” or “non-recombinant” host refers to a wild-type organism that does not contain the heterologous nucleic acid molecule.

By “does not express” means that expression of a protein or gene cannot be detected by standard methods. In the case of cell surface markers, expression can be measured by flow cytometry, using a cut-off values as obtained from negative controls (i.e., cells known to lack the antigen of interest) or by isotype controls (i.e., measuring non-specific binding of the antibody to the cell). Thus, a cell that “does not express” a marker appears similar to the negative control for that marker. For gene expression, a gene “does not express” if the presence of its mRNA cannot be visually detected on a standard agarose gel following standard PCR protocols.

As used herein, the term “endogenous” refers to nucleic acid and/or amino acid sequence naturally occurring in the cell of interest.

As used herein, the term “exogenous” refers to a heterologous nucleic acid and/or amino acid sequence that is not normally found in the cell of interest. For example, a transgene refers to a heterologous nucleic acid sequence that is introduced into a cell of interest by transfection.

As used herein, the term “transfection” refers to the introduction of an exogenous nucleotide sequence, such as DNA vectors in the case of mammalian target cells, into a target cell whether or not any coding sequences are ultimately expressed. Numerous methods of transfection are known to those skilled in the art, such as: chemical methods (e.g., calcium-phosphate transfection), physical methods (e.g., electroporation, microinjection, and particle bombardment), fusion (e.g., liposomes), receptor-mediated endocytosis (e.g., DNA-protein complexes, viral envelope/capsid-DNA complexes), nanoparticles or by transduction with recombinant viruses.

As used herein, the term “construct” refers to a recombinant genetic molecule having one or more isolated polynucleotide sequences. Genetic constructs used for transgene expression in a host organism include in the 5′-3′ direction, a promoter sequence; a sequence encoding a gene of interest; and a termination sequence. The construct may also include selectable marker gene(s) and other regulatory elements for expression.

As used herein, the term “endothelial progenitor cell” refers to precursors of cells in the endothelial cell lineage. Exemplary endothelial progenitor cells include, but are not limited to, colony-forming unit-Hill (CFU-Hill) cells, circulating angiogenic cells (CACs) and endothelial colony-forming cells (ECFCs). CFU-Hill cells and CACs are usually referred to as early outgrowth EPCs whereas ECFCs are termed as late outgrowth EPCs. Although the contribution of endothelial progenitor cells including early outgrowth EPCs and late outgrowth EPCs to new vessel formation has been established, the underlying mechanisms remain unclear.

The disclosure provides for the engineering of pluripotent stem cells and their differentiation into mesodermal (MSD) precursor cells for the treatment of subjects with perfusion disorders.

### Generation and Maintenance of Pluripotent Stem Cells

A “stem cell” is a multipotent or pluripotent cell that (i) is capable of self-renewal; and (ii) can give rise to more than one type of cell through asymmetric cell division. The term “self-renewal” as used herein, refers to the process by which a stem cell divides to generate one (asymmetric division) or two (symmetric division) daughter cells having development potential indistinguishable from the mother cell. Self-renewal involves both proliferation and the maintenance of an undifferentiated state.

Pluripotent stem cells have the ability to give rise to progeny that can undergo differentiation, under the appropriate conditions, into cell types from all three embryonic germ layers (endoderm, mesoderm, and ectoderm) from which all tissues and organs derive. The endoderm is the source of, for example, pharynx, esophagus, stomach, intestine and associated glands (e.g., salivary glands), liver, epithelial linings of respiratory passages and gastrointestinal tract, pancreas and lungs. The mesoderm is the source of, for example, smooth and striated muscle, connective tissue, blood vessels, the cardiovascular system, blood cells, endothelial cells, bone marrow, skeleton, reproductive organs and excretory organs. Ectoderm is the source of, for example, epidermis (epidermal layer of the skin), sensory organs, the entire nervous system, including brain, spinal cord, and all the outlying components of the nervous system.

In contrast, “multipotent cells” can develop into more than one cell type but are more limited than pluripotent cells. Adult stem cells, such as hematopoietic stem cells and cord blood stem cells, are considered multipotent.

Thus, pluripotent stem cells can contribute to many or if not all tissues of a prenatal, postnatal or adult animal. A standard art-accepted test, such as the ability to form a teratoma in 8-12-week-old SCID mice, can be used to establish the pluripotency of a cell population, however identification of various pluripotent stem cell characteristics can also be used to distinguish pluripotent cells from other cells. For example, the ability to give rise to progeny that can undergo differentiation, under the appropriate conditions, into cell types that collectively demonstrate characteristics associated with cell lineages from all of the three germinal layers (endoderm, mesoderm, and ectoderm) is a pluripotent stem cell characteristic. Expression or non-expression of certain combinations of molecular markers are also pluripotent stem cell characteristics.

For example, human pluripotent stem cells express at least some, and optionally all, of the markers from the following non-limiting list: SSEA-3, SSEA-4, TRA-1-60, TRA-1-81, TRA-2-49/6E, ALP, SOX2, E-CADHERIN, UTF-1, OCT4, REX1, AND NANOG.

Pluripotent stem cells suitable for use in the methods of the present disclosure are thus cells with unlimited self-renewal potential in culture including, for example, embryonic stem (ES) cells, primordial germ cells or induced pluripotent stem cells. In a preferred embodiment, pluripotent stem cells are cells that express at least one functional stem cell transcription factor, e.g., Oct-4A, SOX-2 or NANOG. A “functional” stem cell transcription factor does not include pseudogenes of OCT-4, SOX-2 or NANOG.

Examples of pluripotent cells, include, but are not limited to, the human embryonic stem cell (hESC) line H9, fibroblast-derived human iPS cell line DF19-9-11T, hiPS cell line FCB-iPS-1; or hiPS cell line FCB-iPS-2, as described, for example, in the PCT publication WO 2015/138634, the content of which is incorporated by reference herein in its entirety.

In certain embodiments, the pluripotent stem cells can be induced pluripotent stem cells (iPSCs) that are generated by introducing a specific combination of stem cell transcription factors into a non-pluripotent cell (e.g. Oct-3/4, Sox2, KLF4 and c-Myc; see, Takahashi, K. & Yamanaka, S. Cell 126, 663-676 (2006); Okita, K. et al. Nature 448, 313-317 (2007); Wernig, M. et al. Nature 448, 318-324 (2007); Maherali, N. et al. Cell Stem Cell 1, 55-70 (2007); Meissner et al. Nature Biotechnol. 25, 1177-1181 (2007); Yu, J. et al. Science 318, 1917-1920 (2007); Nakagawa, M. et al. Nature Biotechnol. 26, 101-106 (2007); Wernig et al. Cell Stem Cell 2, 10-12 (2008)).

In other embodiments, iPSCs can also be chemically induced from adult somatic cells (see, e.g. U.S. Pat. No. 9,394,524, the content of which is incorporated by reference herein in its entirety).

In still other embodiments, primary human skin fibroblasts can be reprogrammed into induced pluripotent stem cells (iPSCs) by gene editing of the endogenous OCT4, SOX2, KLF4, MYC, and LIN28A promoters and optionally a conserved Alu-motif enriched near genes involved in embryo genome activation (EEA-motif) (Weltner et al. (2018) Nature Communications volume 9, Article number: 2643).

Alternatively, induced pluripotent stem cell lines can be obtained from the ATCC, California Institute for Regenerative Medicine (CIRM) or European Bank for Induced Pluripotent Stem Cells as well as from commercial vendors.

Pluripotent cells are cultured under conditions suitable for maintaining pluripotent cells in an undifferentiated state. Methods for maintaining pluripotent cells in vitro, i.e., in an undifferentiated state, are well known in the art. For example, human ES and iPS cells may be maintained in mTeSR1 complete medium on Matrigel™ in 10 cm² tissue culture dishes at 37° C. and 5% CO2 for about two days. In certain embodiments, the culture medium contains leukemia inhibitory factor, or LIF, an interleukin 6 class cytokine that affects cell growth by inhibiting differentiation.

Additional and/or alternative methods for culturing and/or maintaining pluripotent cells may be used. For example, as the basal culture medium, any of TeSR, mTeSR1 aMEM, BME, BGJb, CMRL 1066, DMEM, Eagle MEM, Fischer's media, Glasgow MEM, Ham, IMDM, Improved MEM Zinc Option, Medium 199 and RPMI 1640, or combinations thereof, may be used for culturing and or maintaining pluripotent cells.

The pluripotent cell culture medium used may contain serum or it may be serum-free. Serum-free refers to a medium comprising no unprocessed or unpurified serum. Serum-free media can include purified blood-derived components or animal tissue-derived components, such as, for example, growth factors. The pluripotent cell medium used may contain one or more alternatives to serum, such as, for example, knockout Serum Replacement (KSR), chemically-defined lipid concentrated (Gibco) or Glutamax (Gibco).

Methods for passaging pluripotent cells are well known in the art. For example, after pluripotent cells are plated, the medium may be changed on days 2, 3, and 4 with cells being passaged on day 5. Generally, once a culture container is 70-100% confluent, the cell mass in the container is split into aggregated cells or single cells by any method suitable for dissociation and the aggregated or single cells are transferred into new culture containers. Cell “passaging” is a well-known technique for keeping cells alive and growing cells in vitro for extended periods of time.

Engineering of Pluripotent Stem Cells and their Progeny

In certain embodiments, the term “engineering” refers to a modification of a cell resulting from the delivery of an agent to that cell. The cell can be, for example, a pluripotent stem cell or any progeny resulting from its differentiation such as a mesodermal precursor cell or other endothelial progenitor cells. The agent can be, for example, a nucleic acid such as an expression vector comprising a transgene, i.e., a DNA sequence encoding a protein (e.g., a therapeutic protein), which is partly or entirely heterologous, i.e., foreign, to the cell, or, is homologous to an endogenous gene of the cell. The agent can be, for example, an expression vector comprising a cDNA sequence, or a genetically engineered gene sequence encoding, for example, a fusion protein. In other embodiments, the expression of a transgene can generate a non-coding RNA, e.g. an RNA interfering molecule or miRNA. In certain embodiments, the miRNA is capable of regulating expression of intracellular growth factors and/or interleukin molecules. In certain embodiments, the miRNA can be overexpressed to regulate the angiogenic, perfusion recovery, and/or arteriogenesis activity of the target cell and/or surrounding tissue. In certain embodiments, the miRNA may be miR-20, miR-29b, miR-93, miR-93-5, miR-126, miR-190, miR-195, miR-200, miR-203, miR-210, miR-101, miR-126, miR-497, miR-503, miR-638, miR-27b, miR-146a, and miR-128. In certain embodiments, the cells may express one or more target miRNA agent molecules. In certain embodiments, the agent is a transducible protein such as a Tat fusion protein.

In certain embodiments, the agent enhances the non-neoplastic proliferation and/or survival of the mesodermal precursor cell in culture and/or the migration of mesodermal precursor cells and their progeny toward ischemic tissue in vivo.

In addition to transcription factors, a number of mechanisms contribute to the regulation of gene expression in endothelial progenitor cells including noncoding RNAs (e.g. microRNAs (miRNAs), small interfering RNAs (siRNAs), piwi-interacting RNAs, small nucleolar RNAs and long non-coding RNAs (lncRNAs)), histone modification (e.g. histone acetylation) and DNA modifications (e.g. DNA methylation), collectively referred to as epigenetic modifications (recently reviewed by Schiattarella et al. Vascular Pharmacology (2018) 107, 43-52). Thus, in certain embodiments, the term “engineering” can refer to a modification of a mesodermal precursor cell through contact with a small molecule that can modulate the expression of one or more endogenous genes. For example, the agent can be a modulator of epigenesis that enhances or inhibits endogenous gene expression. In certain embodiments, the agent can be a small molecule histone deacetylase inhibitor (HDAC inhibitors, HDACi, HDIs) that suppresses histone deacetylase enzymatic activity.

In certain embodiments, the agent comprises a transgene encoding telomerase, the expression of which enhances the non-neoplastic proliferation and/or survival of mesodermal precursor cells. The telomerase coding sequence can be operably linked to a constitutive, induced or tissue-specific promoter according to methods well known in the art.

In certain embodiments, expression of telomerase in mesodermal precursor cells can be induced transiently by the transduction of a transducible recombinant telomerase.

In certain embodiments, expression of telomerase in mesodermal precursor cells can be induced transiently by transfection of mRNAs encoding telomerase.

In certain embodiments, the expression of a telomerase transgene in mesodermal precursor cells enhances their non-neoplastic proliferation in culture by at least about 25%, 50%, 100%, 200%, 300%, 400% or 500% or more as compared to the non-neoplastic proliferation of mesodermal precursor cells transformed with a control transgene that does not encode telomerase.

The telomerase gene (also known as TERT, telomerase reverse transcriptase, telomerase-associated protein, telomerase catalytic subunit, EC 2.7.7.49, HEST2, TCS1, EST2, TP2, TRT, EC 2.7.7 56, PFBMFT1, DKCA2, DKCB4, CMM9 and HTRT) encodes a ribonucleoprotein polymerase that maintains telomere ends by addition of the telomere repeat TTAGGG. The enzyme consists of a protein component with reverse transcriptase activity, encoded by the hTERT gene in human, and an RNA component, known as TERC, which serves as a template for the telomere repeat. Telomerase expression plays a key role in cellular senescence, as it is normally repressed in postnatal somatic cells resulting in progressive shortening of telomeres.

In certain embodiments, the transgene encoding human telomerase (hTERT) comprises the cDNA sequence (NM 1982530) having the DNA sequence of SEQ ID NO: 6 and amino acid sequence of SEQ ID NO: 5 as shown in TABLE I below:

TABLE I

| SEQ | | | |
|---|---|---|---|
| ID NO: | | hTERT cDNA SEQUENCE | |
| 5 | 1 | M P R A P R C R A V R S L L R S H Y R E | 20 |
| 6 | 1 | atgccgcgcgctccccgctgccgagccgtgcgctccctgctgcgcagccactaccgcgag | 60 |
| | 21 | V L P L A T F V R R L G P Q G W R L V Q | 40 |
| | 61 | gtgctgccgctggccacgttcgtgcggcgcctggggccccagggctggcggctggtgcag | 120 |
| | 41 | R G D P A A F R A L V A Q C L V C V P W | 60 |
| | 121 | cgcggggacccggcggctttccgcgcgctggtggcccagtgcctggtgtgcgtgccctgg | 180 |
| | 61 | D A R P P P A A P S F R Q V S C L K E L | 80 |
| | 181 | gacgcacggccgccccccgccgccccctccttccgccaggtgtcctgcctgaaggagctg | 240 |
| | 81 | V A R V L Q R L C E R G A K N V L A F G | 100 |
| | 241 | gtggcccgagtgctgcagaggctgtgcgagcgcggcgcgaagaacgtgctggccttcggc | 300 |
| | 101 | F A L L D G A R G G P P E A F T T S V R | 120 |
| | 301 | ttcgcgctgctggacggggcccgcgggggcccccccgaggccttcaccaccagcgtgcgc | 360 |
| | 121 | S Y L P N T V T D A L R G S G A W G L L | 140 |
| | 361 | agctacctgcccaacacggtgaccgacgcactgcgggggagcggggcgtgggggctgctg | 420 |
| | 141 | L R R V G D D V L V H L L A R C A L F V | 160 |
| | 421 | ctgcgccgcgtgggcgacgacgtgctggttcacctgctggcacgctgcgcgctctttgtg | 480 |
| | 161 | L V A P S C A Y Q V C G P P L Y Q L G A | 180 |
| | 481 | ctggtggctcccagctgcgcctaccaggtgtgcgggccgccgctgtaccagctcggcgct | 540 |
| | 181 | A T Q A R P P P H A S G P R R R L G C E | 200 |
| | 541 | gccactcaggcccggcccccgccacacgctagtggaccccgaaggcgtctgggatgcgaa | 600 |
| | 201 | R A W N H S V R E A G V P L G L P A P G | 220 |
| | 601 | cgggcctggaaccatagcgtcagggaggccggggtccccctgggcctgccagccccgggt | 660 |
| | 221 | A R R R G G S A S R S L P L P K R P R R | 240 |
| | 661 | gcgaggaggcgcgggggcagtgccagccgaagtctgccgttgcccaagaggcccaggcgt | 720 |
| | 241 | G A A P E P E R T P V G Q G S W A H P G | 260 |
| | 721 | ggcgctgcccctgagccggagcggacgcccgttgggcaggggtcctgggcccacccgggc | 780 |
| | 261 | R T R G P S D R G F C V V S P A R P A E | 280 |
| | 781 | aggacgcgtggaccgagtgaccgtggtttctgtgtggtgtcacctgccagacccgccgaa | 840 |
| | 281 | E A T S L E G A L S G T R H S H P S V G | 300 |
| | 841 | gaagccacctctttggagggtgcgctctctggcacgcgccactcccacccatccgtgggc | 900 |
| | 301 | R Q H H A G P P S T S R P P R P W D T P | 320 |
| | 901 | cgccagcaccacgcgggccccccatccacatcgcggccaccacgtccctgggacacgcct | 960 |
| | 321 | C P P V Y A E T K H F L Y S S G D K E Q | 340 |
| | 961 | tgtcccccggtgtacgccgagaccaagcacttcctctactcctcaggcgacaaggagcag | 1020 |
| | 341 | L R P S F L L S S L R P S L T G A R R L | 360 |
| | 1021 | ctgcggccctccttcctactcagctctctgaggcccagcctgactggcgctcggaggctc | 1080 |
| | 361 | V E T I F L G S R P W M P G T P R R L P | 380 |
| | 1081 | gtggagaccatctttctgggttccaggccctggatgccagggactccccgcaggttgccc | 1140 |
| | 381 | R L P Q R Y W Q M R P L F L E L L G N H | 400 |
| | 1141 | cgcctgccccagcgctactggcaaatgcggcccctgtttctggagctgcttgggaaccac | 1200 |
| | 401 | A Q C P Y G V L L K T H C P L R A A V T | 420 |
| | 1201 | gcgcagtgcccctacggggtgctcctcaagacgcactgcccgctgcgagctgcggtcacc | 1260 |
| | 421 | P A A G V C A R E K P Q G S V A A P E E | 440 |
| | 1261 | ccagcagccggtgtctgtgcccgggagaagccccagggctctgtggcggcccccgaggag | 1320 |
| | 441 | E D T D P R R L V Q L L R Q H S S P W Q | 460 |
| | 1321 | gaggacacagacccccgtcgcctggtgcagctgctccgccagcacagcagcccctggcag | 1380 |
| | 461 | V Y G F V R A C L R R L V P P G L W G S | 480 |
| | 1381 | gtgtacggcttcgtgcgggcctgcctgcgccggctggtgcccccaggcctctggggctaa | 1440 |
| | 481 | R H N E R R F L R N T K K F I S L G K H | 500 |
| | 1441 | aggcacaacgaacgccgcttcctcaggaacaccaagaagttcatctccctggggaagcat | 1500 |
| | 501 | A K L S L Q E L T W K M S V R D C A W L | 520 |
| | 1501 | gccaagctctcgctgcaggagctgacgtggaagatgagcgtgcgggactgcgcttggctg | 1560 |
| | 521 | R R S P G V G C V P A A E H R L R E E I | 540 |
| | 1561 | cgcaggagcccaggggttggctgtgttccggccgcagagcaccgtctgcgtgaggagatc | 1620 |
| | 541 | L A K F L H W L M S V Y V V E L L R S F | 560 |
| | 1621 | ctggccaagttcctgcactggctgatgagtgtgtacgtcgtcgagctgctcaggtctttc | 1680 |
| | 561 | F Y V T E T T F Q K N R L F F Y R K S V | 580 |
| | 1681 | ttttatgtcacggagaccacgtttcaaaagaacaggctctttttctaccggaagagtgtc | 1740 |
| | 581 | W S K L Q S I G I R Q H L K R V Q L R E | 600 |
| | 1741 | tggagcaagttgcaaagcattggaatcagacagcacttgaagagggtgcagctgcgggag | 1800 |
| | 601 | L S E A E V R Q H R E A R P A L L T S R | 620 |
| | 1801 | ctgtcggaagcagaggtcaggcagcatcgggaagccaggcccgccctgctgacgtccaga | 1860 |
| | 621 | L R F I P K P D G L R P I V N M D Y V V | 640 |
| | 1861 | ctccgcttcatccccaagcctgacgggctgcggccgattgtgaacatggactacgtcgtg | 1920 |
| | 641 | G A R T F R R E K R A E R L T S R V K A | 660 |
| | 1921 | ggagccagaacgttccgcagagaaaagagggccgagcgtctcacctcgagggtgaaggca | 1980 |
| | 661 | L F S V L N Y E R A R R P G L L G A S V | 680 |
| | 1981 | ctgttcagcgtgctcaactacgagcgggcgcggcgccccggcctcctgggcgcctctgtg | 2040 |
| | 681 | L G L D D I H R A W R T F V L R V R A Q | 700 |
| | 2041 | ctgggcctggacgatatccacagggcctggcgcaccttcgtgctgcgtgtgcgggcccag | 2100 |
| | 701 | D P P P E L Y F V K V D V T G A Y D T I | 720 |
| | 2101 | gacccgccgcctgagctgtactttgtcaaggtggatgtgacgggcgcgtacgacaccatc | 2160 |
| | 721 | P Q D R L T E V I A S I I K P Q N T Y C | 740 |
| | 2161 | ccccaggacaggctcacggaggtcatcgccagcatcatcaaaccccagaacacgtactgc | 2220 |
| | 741 | V R R Y A V V Q K A A H G H V R K A F K | 760 |
| | 2221 | gtgcgtcggtatgccgtggtccagaaggccgcccatgggcacgtccgcaaggccttcaag | 2280 |
| | 761 | S H V S T L T D L Q P Y M R Q F V A H L | 780 |
| | 2281 | agccacgtctctaccttgacagacctccagccgtacatgcgacagttcgtggctcacctg | 2340 |
| | 781 | Q E T S P L R D A V V I E Q S S S L N E | 800 |
| | 2341 | caggagaccagcccgctgagggatgccgtcgtcatcgagcagagctcctccctgaatgag | 2400 |
| | 801 | A S S G L F D V F L R F M C H H A V R I | 820 |
| | 2401 | gccagcagtggcctcttcgacgtcttcctacgcttcatgtgccaccacgccgtgcgcatc | 2460 |
| | 821 | R G K S Y V Q C Q G I P Q G S I L S T L | 840 |
| | 2461 | aggggcaagtcctacgtccagtgccaggggatcccgcagggctccatcctctccacgctg | 2520 |
| | 841 | L C S L C Y G D M E N K L F A G I R R D | 860 |
| | 2521 | ctctgcagcctgtgctacggcgacatggagaacaagctgtttgcggggattcggcgggac | 2580 |
| | 861 | G L L L R L V D D F L L V T P H L T H A | 880 |
| | 2581 | gggctgctcctgcgtttggtggatgatttcttgttggtgacacctcacctcacccacgcg | 2640 |
| | 881 | K T F L R T L V R G V P E Y G C V V N L | 900 |
| | 2641 | aaaaccttcctcaggaccctggtccgaggtgtccctgagtatggctgcgtggtgaacttg | 2700 |
| | 901 | R K T V V N F P V E D E A L G G T A F V | 920 |
| | 2701 | cggaagacagtggtgaacttccctgtagaagacgaggccctgggtggcacggcttttgtt | 2760 |
| | 921 | Q M P A H G L F P W C G L L L D T R T L | 940 |
| | 2761 | cagatgccggcccacggcctattcccctggtgcggcctgctgctggatacccggaccctg | 2820 |
| | 941 | E V Q S D Y S S Y A R T S I R A S L T F | 960 |
| | 2821 | gaggtgcagagcgactactccagctatgcccggacctccatcagagccagtctcaccttc | 2880 |
| | 961 | N R G F K A G R N M R R K L F G V L R L | 980 |
| | 2881 | aaccgcggcttcaaggctgggaggaacatgcgtcgcaaactctttggggtcttgcggctg | 2940 |
| | 981 | K C H S L F L D L Q V N S L Q T V C T N | 1000 |
| | 2941 | aagtgtcacagcctgtttctggatttgcaggtgaacagcctccagacggtgtgcaccaac | 3000 |
| | 1001 | I Y K I L L L Q A Y R F H A C V L Q L P | 1020 |
| | 3001 | atctacaagatcctcctgctgcaggcgtacaggtttcacgcatgtgtgctgcagctccca | 3060 |
| | 1021 | F H Q Q V W K N P T F F L R V I S D T A | 1040 |
| | 3061 | tttcatcagcaagtttggaagaaccccacatttttcctgcgcgtcatctctgacacggcc | 3120 |
| | 1041 | S L C Y S I L K A K N A G M S L G A K G | 1060 |
| | 3121 | tccctctgctactccatcctgaaagccaagaacgcagggatgtcgctgggggccaagggc | 3180 |
| | 1061 | A A G P L P S E A V Q W L C H Q A F L L | 1080 |
| | 3181 | gccgccggccctctgccctccgaggccgtgcagtggctgtgccaccaagcattcctgctc | 3240 |
| | 1081 | K L T R H R V T Y V P L L G S L R T A Q | 1100 |
| | 3241 | aagctgactcgacaccgtgtcacctacgtgccactcctggggtcactcaggacagcccag | 3300 |
| | 1101 | T Q L S R K L P G T T L T A L E A A A N | 1120 |
| | 3301 | acgcagctgagtcggaagctcccggggacgacgctgactgccctggaggccgcagccaac | 3360 |
| | 1121 | P A L P S D F K T I L D * | 1133 |
| | 3361 | ccggcactgccctcagacttcaagaccatcctggactga | 3399 |

In certain embodiments, the transgene encoding human telomerase (hTERT) comprises at least 25 nucleotides of the DNA sequence of SEQ ID NO: 6.

In certain embodiments, the activation of the telomerase is not sufficient for the immortalization of mesodermal precursor cells.

In certain embodiments, the immortalization can be achieved by the concurrent expression of viral genes such as the SV40 large T antigen. Salmon reported a lentiviral approach to induce immortalization by introducing both hTERT and SV40 T antigen into senescent cells (Salmon et al. (2000) Mol. Ther. (4):404-414). However, even though the introduction of viral genes such as the Epstein Barr Virus (EBV), Simian virus 40 (SV40), large T antigen (TAg), Adenovirus E1A and E1B, human papilloma virus (HPV) E6 and E7 have been equally used to permanently immortalize primary cells, such immortalized cells lose the properties of primary cells by inactivating one or another (depending on the cell type) of the above mentioned tumor suppressor genes, which allow cells to re-enter the cell cycle and permanently bypass replicative senescence (see, for example, U.S. Pat. No. 9,670,504, the content of which is incorporated by reference herein in its entirety).

In certain embodiments, the immortalization of mesodermal precursor cells can be achieved by down regulating the translation of an endogenous tumor suppressor gene known to play a role in cell senescence. Such techniques encompass the use of RNA interference molecules directed against one or more tumor suppressor mRNAs. The method of using siRNA and miRNA is well known in the art, e.g. as described by Pei and Tuschl, 2006 (Nat. methods. 3: 670-676 and Chang et al., 2006, Nat. Methods. 3: 707-714.) In a preferred embodiment, the transgene inducing immortalization expresses an shRNA targeting an endogenous tumor suppressor mRNA. In certain embodiments, the shRNA targets a G1-specific tumor suppressor mRNA. In certain embodiments, the shRNA targets a G1-specific tumor suppressor mRNA chosen from the group of p16INK4A, p15INK4B, p18INK4C, p19INK4D, p21Cipl, p27Kip1, or p27Kip2 mRNA.

In certain embodiments, the agent comprises a transgene encoding PSGL-1, the expression of which enhances the migration of mesodermal precursor cells and their progeny toward ischemic tissue in vivo. The PSGL-1 coding sequence can be operably linked to a constitutive, induced or tissue-specific promoter according to methods well known in the art.

In certain embodiments, the expression of the PSGL-1 transgene in mesodermal precursor cells can enhance their migration toward ischemic tissue in vivo by at least about 25%, 50%, 75%, 100%, 200%, 300%, 400% or 500% or more as compared to the migration of mesodermal precursor cells and their progeny transformed with a control transgene that does not encode PSGL-1.

In certain embodiments, expression of PSGL-1 in mesodermal precursor cells can be induced transiently by the transduction of a transducible recombinant PSGL-1.

In certain embodiments, expression of PSGL-1 in mesodermal precursor cells can be induced transiently by transfection of mRNAs encoding PSGL-1.

The P-Selectin Glycoprotein Ligand 1 or PSGL-1 gene (also referred to as selectin P ligand, cutaneous lymphocyte-associated associated antigen, CD162 antigen, CD162 or CLA) is a glycoprotein found on white blood cells and endothelial cells that binds to the cell adhesion molecule, P-selectin.

In certain embodiments, the transgene encoding human P-Selectin Glycoprotein Ligand 1 (PSGL-1) comprises the cDNA sequence (NM 001206609) having the DNA sequence of SEQ ID NO: 2 and amino acid sequence of SEQ ID NO: 1 as shown in TABLE II below:

TABLE II

| SEQ | | | |
|---|---|---|---|
| ID NO | | PSGL-1 cDNA SEQUENCE | |
| 1 | 1 | M A V G A S G L E G D K M A G A M P L Q | 20 |
| 2 | 1 | atggcagtgggggccagtggtctagaaggagataagatggctggtgccatgcctctgcaa | 60 |
| | 21 | L L L L L I L L G P G N S L Q L W D T W | 40 |
| | 61 | ctcctcctgttgctgatcctactgggccctggcaacagcttgcagctgtgggacacctgg | 120 |
| | 41 | A D E A E K A L G P L L A R D R R Q A T | 60 |
| | 121 | gcagatgaagccgagaaagccttgggtcccctgcttgcccgggaccggagacaggccacc | 180 |
| | 61 | E Y E Y L D Y D F L P E T E P P E M L R | 80 |
| | 181 | gaatatgagtacctagattatgatttcctgccagaaacggagcctccagaaatgctgagg | 240 |
| | 81 | N S T D T T P L T G P G T P E S T T V E | 100 |
| | 241 | aacagcactgacaccactcctctgactgggcctggaacccctgagtctaccactgtggag | 300 |
| | 101 | P A A R R S T G L D A G G A V T E L T T | 120 |
| | 301 | cctgctgcaaggcgttctactggcctggatgcaggaggggcagtcacagagctgaccacg | 360 |
| | 121 | E L A N M G N L S T D S A A M E I Q T T | 140 |
| | 361 | gagctggccaacatggggaacctgtccacggattcagcagctatggagatacagaccact | 420 |
| | 141 | Q P A A T E A Q T T Q P V P T E A Q T T | 160 |
| | 421 | caaccagcagccacggaggcacagaccactcaaccagtgcccacggaggcacagaccact | 480 |
| | 161 | P L A A T E A Q T T R L T A T E A Q T T | 180 |
| | 481 | ccactggcagccacagaggcacagacaactcgactgacggccacggaggcacagaccact | 540 |
| | 181 | P L A A T E A Q T T P P A A T E A Q T T | 200 |
| | 541 | ccactggcagccacagaggcacagaccactccaccagcagccacggaagcacagaccact | 600 |
| | 201 | Q P T G L E A Q T T A P A A M E A Q T T | 220 |
| | 601 | caacccacaggcctggaggcacagaccactgcaccagcagccatggaggcacagaccact | 660 |
| | 221 | A P A A M E A Q T T P P A A M E A Q T T | 240 |
| | 661 | gcaccagcagccatggaagcacagaccactccaccagcagccatggaggcacagaccact | 720 |
| | 241 | Q T T A M E A Q T T A P E A T E A Q T T | 260 |
| | 721 | caaaccacagccatggaggcacagaccactgcaccagaagccacggaggcacagaccact | 780 |
| | 261 | Q P T A T E A Q T T P L A A M E A L S T | 280 |
| | 781 | caacccacagccacggaggcacagaccactccactggcagccatggaggccctgtccaca | 840 |
| | 281 | E P S A T E A L S M E P T T K R G L F I | 300 |
| | 841 | gaacccagtgccacagaggccctgtccatggaacctactaccaaaagaggtctgttcata | 900 |
| | 301 | P F S V S S V T H K G I P M A A S N L S | 320 |
| | 901 | cccttttctgtgtcctctgttactcacaagggcattcccatggcagccagcaatttgtcc | 960 |
| | 321 | V N Y P V G A P D H I S V K Q C L L A I | 340 |
| | 961 | gtcaactacccagtgggggccccagaccacatctctgtgaagcagtgcctgctggccatc | 1020 |
| | 341 | L I L A L V A T I F F V C T V V L A V R | 360 |
| | 1021 | ctaatcttggcgctggtggccactatcttcttcgtgtgcactgtggtgctggcggtccgc | 1080 |
| | 361 | L S R K G H M Y P V R N Y S P T E M V C | 380 |
| | 1081 | ctctcccgcaagggccacatgtaccccgtgcgtaattactcccccaccgagatggtctgc | 1140 |
| | 381 | I S S L L P D G G E G P S A T A N G G L | 400 |
| | 1141 | atctcatccctgttgcctgatgggggtgaggggccctctgccacagccaatgggggcctg | 1200 |
| | 401 | S K A K S P G L T P E P R E D R E G D D | 420 |
| | 1201 | tccaaggccaagagcccgggcctgacgccagagcccagggaggaccgtgagggggatgac | 1260 |
| | 421 | L T L H S F L P * | 429 |
| | 1261 | ctcaccctgcacagcttcctcccttag | 1287 |

In certain embodiments, the transgene encoding human P-Selectin Glycoprotein Ligand 1 (PSGL-1) comprises at least 25 nucleotides of the DNA sequence of SEQ ID NO: 2.

In certain embodiments, the agent comprises a transgene encoding neuropilin-1, the expression of which enhances the migration of mesodermal precursor cells and their progeny toward ischemic tissue in vivo. The neuropilin-1 coding sequence can be operably linked to a constitutive, induced or tissue-specific promoter according to methods well known in the art.

In certain embodiments, the expression of the neuropilin-1 transgene in mesodermal precursor cells can enhance their migration toward ischemic tissue in vivo by at least about 25%, 50%, 75%, 100%, 200%, 300%, 400% or 500% or more as compared to the migration of mesodermal precursor cells and their progeny transformed with a control transgene that does not encode neuropilin-1.

In certain embodiments, expression of neuropilin-1 in mesodermal precursor cells can be induced transiently by the transduction of a transducible recombinant neuropilin-1.

In certain embodiments, expression of neuropilin-1 in mesodermal precursor cells can be induced transiently by transfection of mRNAs encoding neuropilin-1.

Neuropilin-1 (also referred to as Neuropilin, Vascular Endothelial Cell Growth Factor 165 Receptor, VEGF165R, NRP, Transmembrane Receptor 3, CD304 Antigen, BDCA4, CD304 or NP1) contains specific protein domains which allow it to participate in several different types of signaling pathways that control cell migration. Neuropilins contain a large N-terminal extracellular domain, made up of complement-binding, coagulation factor V/VIII, and meprin domains. These proteins also contain a short membrane-spanning domain and a small cytoplasmic domain. Neuropilins bind many ligands and various types of co-receptors; they affect cell survival, migration, and attraction. Some of the ligands and co-receptors bound by neuropilins are vascular endothelial growth factor (VEGF) and semaphorin family members. Several alternatively spliced transcript variants that encode different protein isoforms have been reported.

In certain embodiments, the transgene encoding human neuropilin-1 (NRP-1) comprises the cDNA sequence (NM 003873) having the DNA sequence of SEQ ID NO: 4 and amino acid sequence of SEQ ID NO: 3 as shown in TABLE III below:

TABLE III

| SEQ | | | |
|---|---|---|---|
| ID NO: | | NEUROPILIN-1 cDNA SEQUENCE | |
| 3 | 1 | M E R G L P L L C A V L A L V L A P A G | 20 |
| 4 | 1 | atggagagggggctgccgctcctctgcgccgtgctcgccctcgtcctcgccccggccggc | 60 |
| | 21 | A F R N D K C G D T I K I E S P G Y L T | 40 |
| | 61 | gcttttcgcaacgataaatgtggcgatactataaaaattgaaagccccgggtaccttaca | 120 |
| | 41 | S P G Y P H S Y H P S E K C E W L I Q A | 60 |
| | 121 | tctcctggttatcctcattcttatcacccaagtgaaaaatgcgaatggctgattcaggct | 180 |
| | 61 | P D P Y Q R I M I N F N P H F D L E D R | 80 |
| | 181 | ccggacccataccagagaattatgatcaacttcaaccctcacttcgatttggaggacaga | 240 |
| | 81 | D C K Y D Y V E V F D G E N E N G H F R | 100 |
| | 241 | gactgcaagtatgactacgtggaagtcttcgatggagaaaatgaaaatggacattttagg | 300 |
| | 101 | G K F C G K I A P P P V V S S G P F L F | 120 |
| | 301 | ggaaagttctgtggaaagatagcccctcctcctgttgtgtcttcagggccatttcttttt | 360 |
| | 121 | I K F V S D Y E T H G A G F S I R Y E I | 140 |
| | 361 | atcaaatttgtctctgactacgaaacacatggtgcaggattttccatacgttatgaaatt | 420 |
| | 141 | F K R G P E C S Q N Y T T P S G V I K S | 160 |
| | 421 | ttcaagagaggtcctgaatgttcccagaactacacaacacctagtggagtgataaagtcc | 480 |
| | 161 | P G F P E K Y P N S L E C T Y I V F A P | 180 |
| | 481 | cccggattccctgaaaaatatcccaacagccttgaatgcacttatattgtctttgcgcca | 540 |
| | 181 | K M S E I I L E F E S F D L E P D S N P | 200 |
| | 541 | aagatgtcagagattatcctggaatttgaaagctttgacctggagcctgactcaaatcct | 600 |
| | 201 | P G G M F C R Y D R L E I W D G F P D V | 220 |
| | 601 | ccaggggggatgttctgtcgctacgaccggctagaaatctgggatggattccctgatgtt | 660 |
| | 221 | G P H I G R Y C G Q K T P G R I R S S S | 240 |
| | 661 | ggccctcacattgggcgttactgtggacagaaaacaccaggtcgaatccgatcctcatcg | 720 |
| | 241 | G I L S M V F Y T D S A I A K E G F S A | 260 |
| | 721 | ggcattctctccatggttttttacaccgacagcgcgatagcaaaagaaggtttctcagca | 780 |
| | 261 | N Y S V L Q S S V S E D F K C M E A L G | 280 |
| | 781 | aactacagtgtcttgcagagcagtgtctcagaagatttcaaatgtatggaagctctgggc | 840 |
| | 281 | M E S G E I H S D Q I T A S S Q Y S T N | 300 |
| | 841 | atggaatcaggagaaattcattctgaccagatcacagcttcttcccagtatagcaccaac | 900 |
| | 301 | W S A E R S R L N Y P E N G W T P G E D | 320 |
| | 901 | tggtctgcagagcgctcccgcctgaactaccctgagaatgggtggactcccggagaggat | 960 |
| | 321 | S Y R E W I Q V D L G L L R F V T A V G | 340 |
| | 961 | tcctaccgagagtggatacaggtagacttgggccttctgcgctttgtcacggctgtcggg | 1020 |
| | 341 | T Q G A I S K E T K K K Y Y V K T Y K I | 360 |
| | 1021 | acacagggcgccatttcaaaagaaaccaagaagaaatattatgtcaagacttacaagatc | 1080 |
| | 361 | D V S S N G E D W I T I K E G N K P V L | 380 |
| | 1081 | gacgttagctccaacggggaagactggatcaccataaaagaaggaaacaaacctgttctc | 1140 |
| | 381 | F Q G N T N P T D V V V A V F P K P L I | 400 |
| | 1141 | tttcagggaaacaccaaccccacagatgttgtggttgcagtattccccaaaccactgata | 1200 |
| | 401 | T R F V R I K P A T W E T G I S M R F E | 420 |
| | 1201 | actcgatttgtccgaatcaagcctgcaacttgggaaactggcatatctatgagatttgaa | 1260 |
| | 421 | V Y G C K I T D Y P C S G M L G M V S G | 440 |
| | 1261 | gtatacggttgcaagataacagattatccttgctctggaatgttgggtatggtgtctgga | 1320 |
| | 441 | L I S D S Q I T S S N Q G D R N W M P E | 460 |
| | 1321 | cttatttctgactcccagatcacatcatccaaccaaggggacagaaactggatgcctgaa | 1380 |
| | 461 | N I R L V T S R S G W A L P P A P H S Y | 480 |
| | 1381 | aacatccgcctggtaaccagtcgctctggctgggcacttccacccgcacctcattcctac | 1440 |
| | 481 | I N E W L Q I D L G E E K I V R G I I I | 500 |
| | 1441 | atcaatgagtggctccaaatagacctgggggaggagaagatcgtgaggggcatcatcatt | 1500 |
| | 501 | Q G G K H R E N K V F M R K F K I G Y S | 520 |
| | 1501 | cagggtgggaagcaccgagagaacaaggtgttcatgaggaagttcaagatcgggtacagc | 1560 |
| | 521 | M N G S D W K M I M D D S K R K A K S F | 540 |
| | 1561 | aacaacggctcggactggaagatgatcatggatgacagcaaacgcaaggcgaagtctttt | 1620 |
| | 541 | E G N N N Y D T P E L R T F P A L S T R | 560 |
| | 1621 | gagggcaacaacaactatgatacacctgagctgcggacttttccagctctctccacgcga | 1680 |
| | 561 | F I R I Y P E R A T H G G L G L R M E L | 580 |
| | 1681 | ttcatcaggatctaccccgagagagccactcatggcggactggggctcagaatggagctg | 1740 |
| | 581 | L G C E V E A P T A G P T T P N G N L V | 600 |
| | 1741 | ctgggctgtgaagtggaagcccctacagctggaccgaccactcccaacgggaacttggtg | 1800 |
| | 601 | D E C D D D Q A N C H S G T G D D F Q L | 620 |
| | 1801 | gatgaatgtgatgacgaccaggccaactgccacagtggaacaggtgatgacttccagctc | 1860 |
| | 621 | T G G T T V L A T E K P T V I D S T I Q | 640 |
| | 1861 | acaggtggcaccactgtgctggccacagaaaagcccacggtcatagacagcaccatacaa | 1920 |
| | 641 | S E F P T Y G F N C E F G W G S H K T F | 660 |
| | 1921 | tcagagtttccaacatatggttttaactgtgaatttggctggggctctcacaagaccttc | 1980 |
| | 661 | C H W E H D N H V Q L K W S V L T S K T | 680 |
| | 1981 | tgccactgggaacatgacaatcacgtgcagctcaagtggagtgtgttgaccagcaagacg | 2040 |
| | 681 | G P I Q D H T G D G N F I Y S Q A D E N | 700 |
| | 2041 | ggacccattcaggatcacacaggagatggcaacttcatctattcccaagctgacgaaaat | 2100 |
| | 701 | Q K G K V A R L V S P V V Y S Q N S A H | 720 |
| | 2101 | cagaagggcaaagtggctcgcctggtgagccctgtggtttattcccagaactctgcccac | 2160 |
| | 721 | C M T F W Y H M S G S H V G T L R V K L | 740 |
| | 2161 | tgcatgaccttctggtatcacatgtctgggtcccacgtcggcacactcagggtcaaactg | 2220 |
| | 741 | R Y Q K P E E Y D Q L V W M A I G H Q G | 760 |
| | 2221 | cgctaccagaagccagaggagtacgatcagctggtctggatggccattggacaccaaggt | 2280 |
| | 761 | D H W K E G R V L L H K S L K L Y Q V I | 780 |
| | 2281 | gaccactggaaggaagggcgtgtcttgctccacaagtctctgaaactttatcaggtgatt | 2340 |
| | 781 | F E G E I G K G N L G G I A V D D I S I | 800 |
| | 2341 | ttcgagggcgaaatcggaaaaggaaaccttggtgggattgctgtggatgacattagtatt | 2400 |
| | 801 | N N H I S Q E D C A K P A D L D K K N P | 820 |
| | 2401 | aataaccacatttcacaagaagattgtgcaaaaccagcagacctggataaaaagaaccca | 2460 |
| | 821 | E I K I D E T G S T P G Y E G E G E G D | 840 |
| | 2461 | gaaattaaaattgatgaaacagggagcacgccaggatacgaaggtgaaggagaaggtgac | 2520 |
| | 841 | K N I S R K P G N V L K T L D P I L I T | 860 |
| | 2521 | aagaacatctccaggaagccaggcaatgtgttgaagaccttagaccccatcctcatcacc | 2580 |
| | 861 | I I A M S A L G V L L G A V C G V V L Y | 880 |
| | 2581 | atcatagccatgagtgccctgggggtcctcctgggggctgtctgtggggtcgtgctgtac | 2640 |
| | 881 | C A C W H N G M S E R N L S A L E N Y N | 900 |
| | 2641 | tgtgcctgttggcataatgggatgtcagaaagaaacttgtctgccctggagaactataac | 2700 |
| | 901 | F E L V D G V K L K K D K L N T Q S T Y | 920 |
| | 2701 | tttgaacttgtggatggtgtgaagttgaaaaaagacaaactgaatacacagagtacttat | 2760 |
| | 921 | S E A * | 924 |
| | 2761 | tcggaggcatga | 2772 |

In certain embodiments, the transgene encoding human neuropilin-1 (NRP-1) comprises at least 25 nucleotides of the DNA sequence of SEQ ID NO: 4.

In certain embodiments, the expression of the neuropilin-1 transgene in mesodermal precursor cells can enhance their migration toward ischemic tissue in vivo by at least about 25%, 50%, 75%, 100%, 200%, 300%, 400% or 500% or more as compared to the migration of mesodermal precursor cells and their progeny transformed with a control transgene that does not encode neuropilin.

In certain embodiments, the transgene can encode a reporter protein, for example, cell-surface markers and bioluminescent (luciferase) or fluorescent proteins (e.g., green fluorescent protein (GFP), enhanced green fluorescent protein (EGFP), yellow fluorescent protein (YFP), blue fluorescent protein (BFP) and red fluorescent protein (RFP), e.g. tdTomato RFP (recently reviewed by Senutovitch et al. Exp. Biol. Med. (2015); 240(6): 795-808, which is incorporated by reference herein in its entirety). In other embodiments, the transgene can encode a drug resistance gene.

In certain embodiments, the transgene comprises a coding region that is operably linked to one or more transcription regulatory sequences such as promoters in combination with other nucleic acid elements, such as introns, poly A sites and/or locus control regions (LCR), that may be necessary for optimal expression of the selected nucleic acid sequence.

As used herein, the phrase “operably linked” when referring to a transcription regulatory element and a coding sequence is intended to mean that the regulatory sequence is associated with the coding sequence in such a manner as to facilitate the transcription of the coding sequence.

As used herein, the term “promoter” refers generally to proximal promoters found in the 5′ flanking region of protein-coding genes that facilitates the binding of transcription factors required for their transcription by RNA polymerase II. In certain embodiments, the promoter may further comprise an enhancer and other position independent cis-acting regulatory elements that enhance transcription from the proximal promoter such as scaffold/matrix attachment region (S/MAR) element. In certain embodiments, genes transcribed by RNA polymerase III can have their promoter located within the gene itself, i.e. downstream of the transcription start site.

In certain embodiments, the transgene may comprise a protein-coding region operably linked to either a constitutive, inducible or tissue-specific promoter.

Exemplary embodiments of constitutive promoters include, but are not limited to, viral promoters from polyoma, adenovirus, cytomegalovirus (CMV) and simian virus 40 (SV40). In an exemplary configuration, the protein coding sequences are flanked upstream (i.e., 5′) by the human cytomegalovirus IE promoter and downstream (i.e., 3′) by an SV40 poly(A) signal. The human cytomegalovirus IE promoter is described in Boshart et al. (1985) Cell 41:521 530, which is incorporated by reference herein in its entirety. Other ubiquitously expressing promoters which can be used include the HSV-TK promoter, β-actin promoters, CBh promoter and the EF-1α promoter.

Exemplary embodiments of inducible expression systems include, but are not limited to: a tetracycline (Tet) inducible system (see e.g., Gossen et al. (1992) Proc. Natl. Acad. Sci. USA 89:5547 5551; Gossen et al. (1995) Science 268:1766 1769, which are incorporated by reference herein in their entireties); a FK506/rapamycin inducible system (see e.g., Spencer et al. (1993) Science 262:1019 1024; Belshaw et al. (1996) Proc. Natl. Acad. Sci. USA 93:4604 4607, which are incorporated by reference herein in their entireties); a RU486/mifepristone inducible system (Wang et al. Proceedings of the National Academy of Sciences (1994) 91(17):8180-4, which is incorporated by reference herein in its entirety); a cumate inducible system (Mullick et al. BMC Biotechnol. 2006 Nov. 3; 6:43, which is incorporated by reference herein in its entirety) or an ecdysone inducible system (for review, see Rossi et al. (1989) Curr. Op. Biotech. 9:451 456, which is incorporated by reference herein in its entirety). Many constitutive, tissue-specific and inducible promoters are now also commercially available from vendors such as Origene, Promega, Invitrogen, System Biosciences and Invivogen.

In certain embodiments, the term “inducible” means the transcription of a protein-coding sequence can be regulated by an inducer or repressor molecule acting on one or more transcription factors binding to its promoter. For example, removal of the inducer down-regulates transgene expression whereas the presence of the inducer up-regulates transgene expression. Conversely, removal of a repressor up-regulates transgene expression whereas the presence of the repressor down-regulates transgene expression.

In other embodiments, the expression of a protein-coding sequence can be down-regulated by site-specific recombinase mediated excision of the transgene or a portion thereof.

In certain embodiments, the transgenes disclosed herein can be fused in frame to sequences encoding destabilizing domains (DD), e.g., FK506- and rapamycin-binding protein (FKBP12). that destabilize the resulting fusion proteins. The level of the fusion protein can then be regulated through the addition of the small-molecule rapamycin. In the absence of the small molecule the fusion protein is destabilized and degraded. Expression of the fusion protein can then be regulated by the small molecule in a dose-dependent manner.

Small-Molecule Modulation of Protein Homeostasis is reviewed by Burslem and Crews Chem. Rev. (2017) 117, 11269-11301, the content of which is incorporated by reference herein in its entirety.

Methods of delivering transgenes into cells are well known in the art.

In certain embodiments, the transgene can be transfected into cells as part of an episomal vector or expression cassette which is able to replicate independently without the need to integrate in the genome of the host cell. The transgene exists in parallel with the genome of the host cell and is replicated during the cell cycle whereby in the course of this the transgene is copied, depending on the number of copies present before and after cell division and whereby the said copies of the transgene are distributed statistically amongst the resulting cells. Exemplary episomal plasmids include, but are not limited to, constructs having sequences from DNA viruses, such as BK virus, bovine papilloma virus 1 and Epstein-Barr virus.

In certain embodiments, the transgene can be inserted into a viral vector, e.g., a lentiviral vector, or a plasmid and transfected into cells by electroporation, calcium phosphate precipitation, nanoparticles or liposomes etc. where it becomes randomly integrated into the cell's own genome.

In recent years, a strategy for transgene integration has been developed that uses cleavage with site-specific nucleases for targeted insertion into a chosen genomic locus (see below, and e.g., U.S. Pat. No. 7,888,121, the content of which is incorporated by reference herein in its entirety). Nucleases specific for targeted genes can be utilized such that the transgene construct is inserted by either homology directed repair (HDR) or by end capture during non-homologous end joining (NHEJ) driven processes.

In yet another embodiment, the insertion of the transgene can be targeted to specific gene sequence within the genome using homologous recombination producing a “knock-out” where the insertion disrupts the function of the targeted gene or a “knock-in” where the targeted gene function is not altered.

In preferred embodiments, the transgene can be inserted into a genomic safe harbor site (GSH), i.e., a site in the genome that is able to accommodate the integration of new genetic material in a manner that ensures that the newly inserted genetic elements: (i) function predictably and (ii) do not cause alterations of the host genome posing a risk to the host cell or organism (recently reviewed by Papapetrou et al., Mol Ther. (2016) 24(4): 678-684).

Exemplary “safe harbor” loci include, but are not limited to, (i) the adeno-associated virus site 1 (AAVS1), a naturally occurring site of integration of AAV virus on chromosome 19; (ii) the chemokine (C-C motif) receptor 5 (CCR5) gene, a chemokine receptor gene known as an HIV-1 coreceptor; and (iii) the human ortholog of the mouse Rosa26 locus, a locus extensively validated in the murine setting for the insertion of ubiquitously expressed transgenes (see, e.g., U.S. Pat. Nos. 7,888,121; 7,972,854; 7,914,796; 7,951,925; 8,110,379; 8,409,861; 8,586,526; U.S. Patent Publication Nos. 2003/0232410; 2005/0208489; 2005/0026157; 2006/0063231; 2008/0159996; 2010/00218264; 2012/0017290; 2011/0265198; 2013/0137104; 2013/0122591; 2013/0177983; and 2013/0177960, the contents of which are incorporated by reference herein in their entireties). Other GSH sites include HPRT (but on the X chromosome), Hipp11, TIGRE loci.

In certain embodiments, the exemplary genomic safe harbor can be within the Citrate Lyase Beta-Like (CLYBL) gene (see Example 4; Cerbini et al., PLoS One. 2015; 10(1): e0116032).

### Engineering Pluripotent Stem Cells by Gene Editing

Gene editing refers to methods of modifying DNA sequences using site-specific nucleases. including, but not limited to, transcription activator-like effector nucleases (TALENs), meganucleases, zinc-finger nucleases (ZFN) and the clustered regularly interspaced short palindromic repeats (CRISPR)/CRISPR-associated (Cas) systems. See, for example, Urnov et al., (2010) Nature 435(7042):646-51; U.S. Pat. Nos. 8,586,526; 6,534,261; 6,599,692; 6,503,717; 6,689,558; 7,067,317; 7,262,054 and 8,697,359, the contents of which are incorporated by reference herein in their entireties.

Generally, site-specific nucleases act by introducing double strand breaks (DSBs) at desired genomic loci, thereby triggering the endogenous DNA repair machinery. Processing of DSBs by the error-prone nonhomologous end-joining (NHEJ) pathway leads to small insertions and deletions (Indels) useful for generating loss-of-function mutations, whereas error-free homology directed repair (HDR) enables targeted integration of exogenously provided DNA sequences for introducing precise nucleotide (nt) alterations or knock in reporters.

In particular, recent studies have successfully adapted the prokaryotic type II CRISPR (clustered regularly interspaced short palindromic repeat)/Cas system for genome editing in eukaryotic systems. The type II CRISPR/Cas system requires two components: the DNA endonuclease Cas9 protein for DNA cleavage and a variable CRISPR RNA (crRNA) and trans-activating crRNA (tracrRNA) duplex for DNA target recognition. Binding of crRNA/tracrRNA to the target sequence via Watson-Crick base pairing directs Cas9 to any genomic locus of interest for site-specific DNA cleavage.

The requirement of the crRNA-tracrRNA complex can be avoided by use of an engineered “single-guide RNA” (sgRNA) that comprises the hairpin normally formed by the annealing of the crRNA and the tracrRNA (see Jinek et al., (2012) Science 337:816 and Cong et al., (2013) Science 339(6121): 819-823). The sgRNA targets the Cas9 nuclease to the complementary 20 nucleotide (nt) genomic region harboring a 5′-NGG-3′ protospacer-adjacent motif (PAM) (see Ramalingam et al., (2013) Stem Cells and Development 22(4):595-610). The double-stranded DNA breaks generated by Cas9 are then repaired by nonhomologous end-joining (NHEJ) or homology-directed repair (HDR). The CRISPR/Cas system has also been further improved for use in mammalian systems through Cas9 codon optimization.

Nuclease-mediated integration offers the prospect of improved transgene expression, increased safety and expressional durability, as compared to classic integration approaches that rely on random integration of the transgene, since it allows exact transgene positioning for example, at a genomic safe harbor site.

In certain embodiments, a transgene can be engineered to have an “Internal Ribosomal Entry Site” or IRES site upstream of a protein coding region that provides a ribosomal binding for the cap-independent translation of the protein-coding region. Such promoter less IRES-transgenes can then be knocked into, for example, the 3′ untranslated region (3′-UTR) of a target gene. Expression of the transgene then occurs simultaneously with the expression of the target gene (see Examples 1 and 2).

### Engineering Transgenic Pluripotent Stem Cells Using Site-Specific Recombinases

Transgenes introduced into pluripotent stem cells by, for example, gene editing can be further modified by site-specific recombinases effectuating the site-specific recombination between compatible sequence-specific recombination sites. Examples of site-specific recombinase include, without limitation, bacteriophage P1 Cre recombinase that recognizes loxP recombination sites whereas yeast FLP recombinase recognizes FRT recombination sites including any derivatives of a naturally occurring Cre or FLP recombinase that retain the ability to effectuate recombination between two compatible lox sites (see, e.g., Hoess et al., Proc. Natl. Acad. Sci. USA 79:3398-3402 (1982); Sauer, B. L., U.S. Pat. No. 4,959,317, and Hamilton, D. L., et al., J. Mol. Biol. 178:481-486 (1984), the contents of which are incorporated by reference herein in their entireties).

As used herein, the term “FRT site” refers to any art-recognized yeast FRT recombination site, or variant thereof, which includes a 34-base pair FRT site, in which a spacer region of 8 base pairs is flanked by two inverted repeats of 13 base pairs. See, for example, Jayaram et al., Proc. Natl. Acad. Sci. 82, 5875-5879 (1985); Umlauf S. W. et al., EMBO Journal, 7, 1845-1852 (1988); Lee J. et al., EMBO Journal, 18, 784-791, 1999, which are incorporated by reference herein in their entireties. Examples of non-cross-reactive compatible pairs of mutant FRT sites are disclosed, for example, in U.S. Pat. Nos. 7,476,539 and 7,736,897, which are incorporated by reference herein by reference in their entireties.

As used herein, the term “lox P site” refers to any art-recognized lox recombination site, or variant thereof, which includes the 34 base pair loxP site in bacteriophage P1 as well as a number of variant lox sites including, but not limited to, Lox 511, Lox 5171, Lox 2272, M2, M3, M7, M11, Lox71 and Lox66 (Missirlis et al. BMC Genomics 7: 73. 1471-2164, which is incorporated by reference herein in its entirety). Examples of non-cross-reactive compatible pairs of mutant lox sites are disclosed in U.S. Pat. Nos. 7,696,335; 7,060,499 and 7,696,335, the contents of which are incorporated by reference herein by reference in their entireties.

Recombination products are dependent on the location and relative orientation of the recombination sites. When two recombination sites having an identical orientation exist within the same DNA molecule, a DNA sequence flanked by the two recombination sites can be excised by the sequence-specific recombinase to form a circular molecule (excision reaction). Conversely, when two recombination sites exist in different DNA molecules, one of which is a circular DNA, the circular DNA can be inserted into the other DNA molecule via the recombination sites (insertion reaction). In another embodiment, the site-specific recombinases can be optimized. See, for example, International Patent Application Publication No. WO 2014158593 and U.S. Patent Application Publication No. 2010/0050279, which is incorporated by reference herein in its entirety.

In certain embodiments, a transgene, for example, a fluorescent protein reporter expression vector that is knocked into a GSH site can be excised by the transient expression of Cre recombinase, for example, by transducing the cells with Tat-Cre. Cell clones in which the transgene has been excised are then readily identified by the extinction of fluorescence from the reporter (see Example 2).

### Protein Transduction

In certain embodiments, expression of a protein encoded by a transgene in mesodermal precursor cells can be induced transiently by the delivery of a transducible protein encoded by the transgene or the delivery of an mRNA encoding the transgene. A protein can be rendered transducible by fusion to a protein transduction domain. As used herein, the term “transducible protein” refers to a recombinant protein that is conjugated either covalently or non-covalently to a protein transduction domain or PTD.

Protein transduction domains (PTDs), also known as cell penetrating peptides, are a class of small peptides capable of penetrating the plasma membrane of mammalian cells. PTDs can be classified into 3 types: (1) cationic peptides of 6-12 amino acids in length, comprised predominantly of arginine, ornithine and/or lysine residues; (2) hydrophobic peptides such as leader sequences of secreted growth factors and cytokines; and (3) cell-type specific peptides, identified by screening of peptide phage display libraries. Additional PTDs and methods of using same can be found in the published U.S. Patent Applications 2010/0004165 and 2012/0190107, the contents of which is incorporated by reference herein in its entirety.

Methods of administering a transducible protein to cultured cells are described in e.g. WO2000034308 and WO2002055684, the contents of which are incorporated by reference herein in their entireties. In certain embodiments, cultured cells can be transduced with a Tat-fusion protein by simply incubating culture cells with a recombinant transducible protein for 30-60 mins.

In certain embodiments, the transducible protein may further comprise a nuclear localization sequence. Nuclear localization sequences (NLSs) fall into three classes. Two of these are highly basic in nature, those displaying homology to the well-characterized SV40 large T antigen of basic amino acids (PKKKRKV; SEQ ID NO: 7) and bipartite NLSs which contain two stretches of basic amino acids separated by a spacer of 10-12 aa (Rob-bins et al., Cell 64 (1991) 615-623), e.g. that of nucleoplasmin (KRpaatkkagqaKKKK; SEQ ID NO: 8). The third class of NLSs include those resembling the yeast homeodomain containing protein Mata2 (Hall et al., PNAS 87, 6954-6958 (1990)) or the protooncogene c-myc (Makkerh et al., Curr. Biol. 6 (1996) 1025-1027).

Table IV below discloses exemplary PTDs (reproduced from Gagat et al. Int J Mol Med. 2017 December; 40(6): 1615-1623).

TABLE IV

| PROTEIN | PROTEIN | AMINO ACID | SEQ | |
|---|---|---|---|---|
| GROUP | NAME | SEQUENCE | ID NO. | CHARACTERISTICS |
| NATURAL | Tat | GRKKRRQRRRPPQ | 9 | Transcriptional regulator of HIV |
| | pVEC | LLIILRRRIRKQAH | 10 | pVEC, 18 amino acid cell penetrating |
| | | AHSK | | peptide (CPP) derived from murine |
| | | | | vascular endothelial cadherin |
| CHIMERIC | Transportan | GWTLNSAGYLLG | 11 | Protein formed by the combination of |
| | | KINLKALAALAKKIL | | neuropeptide galanin and wasp's |
| | | | | botulinum toxin, mastoparan |
| | MPG | GALFLGFLGAAGST | 12 | Protein obtained by the fusion of the |
| | | MGAWSQPKKKRKV | | transmembrane glycoprotein of HIV, |
| | | | | gp41, with SV40 virus T-antigen |
| | Synthetic | KLALKLALKALKAALK | 13 | Model Amphipathic Protein (MAP) |
| | MAP | LA | | created de novo from lysine, arginine |
| | | | | and leucine residues |
| | R6W3 | RRWWRRWRR | 14 | Artificial peptide created de novo |
| | | | | based on the structure of penetrin |
| CATIONIC | R9 | RRRRRRRRR | 15 | Synthetic sequence of nine arginines |
| | Antp | RQIKIWFQNRRMKWKK | 16 | Homeobox gene of Drosophila |
| | | | | melanogaster |
| HYDROPHOBIC | VP22 | DAATATRGRSAASRPTE | 17 | A component of a capsid of HSV-1 |
| | | RPRAPARSASRPRRVD | | virus |
| | K-FGF | AAVLLPVLLAAP | 18 | Artificial peptide |
| AMPHIPATHIC | VT5 | DPKGDPKGVTVTVTVT | 19 | Capsid protein of rotaviruses |
| | | VTGKGDPKPD | | |
| | SynB1 | RGGRLSYSRRRFSTSTGR | 20 | The peptide derived from protegrin |
| | | | | that can cross the blood-brain barrier. |

In certain embodiments, a site-specific recombinase Cre can be delivered to a cell as a chimeric protein, e.g., a Tat-Cre fusion protein (Joshi et al. Genesis (2002) 33:48-54; Peitz et al., (2002) Proc. Natl. Acad. Sci. USA 99:4489-94, the contents of which are incorporated by reference herein in their entireties). A TAT-Cre has been shown to induce greater than 95% recombination efficiency in fibroblasts and murine embryonic stem cells in vitro.

In certain embodiments, mesodermal (MSD) precursor cells can be immortalized transiently by adding the transducible VP22-hTERT in the culture media to induce proliferation whereas removal of VP22-hTERT would slow or stop proliferation. This approach is well suited to cell therapy applications because the transduced MSD population is not genetically modified. See, for example, the published U.S. Patent Applications 2010/0047218 and 2014/0178965 the contents of which are incorporated by reference herein in their entireties.

mRNA Transduction

In certain embodiments, transgenes can be delivered to mesodermal precursor cells by transfection of a synthetic messenger ribonucleic acid (mRNA) comprising one or more modified nucleosides. Methods of generating modified mRNAs and transfecting same are well known in the art. See, for example, U.S. Pat. No. 9,283,287, the contents of which are incorporated by reference herein in their entireties. In certain embodiments, the mRNA may comprise two coding regions where an IRES element located 3′ to the first coding region is able to elicit the cap independent translation of a second coding region.

### Production and Selection of Mesodermal Precursor Cells

A key advantage of using iPSCs is the ability to identify and isolate very early endothelial progenitor cell populations that would be otherwise inaccessible.

The present disclosure describes a mesodermal precursor cell population obtained through the differentiation of engineered induced pluripotent stem cells (iPSCs) toward the endothelial cell lineage whereby the modifications made improve the survival and clonal proliferation of the mesodermal precursor cell population (see published U.S. Patent Application No. 2017/0022476, the content of which is hereby incorporated by reference in its entirety).

In certain embodiments, the present disclosure also provides a method for generating an isolated population of human KDR⁺NCAM⁺APLNR⁺ mesodermal precursor cells from human pluripotent stem cells. The method comprises providing pluripotent stem cells (PSCs); inducing the pluripotent stem cells to undergo mesodermal differentiation, wherein the mesodermal induction comprises: i) culturing the pluripotent stem cells for about 24 hours in a mesoderm differentiation medium comprising an effective amount of Activin A, vascular endothelial growth factor (VEGF), basic fibroblast growth factor (FGF-2) and bone morphogenetic protein 4 (BMP-4); and ii) replacing the medium of step i) with a mesoderm differentiation medium comprising an effective amount of BMP-4, VEGF and FGF-2 about every 24-48 hours thereafter for about 72 hours; and isolating from the cells induced to undergo mesoderm differentiation, wherein their isolation comprises: iii) selecting for KDR⁺NCAM⁺APLNR⁺ mesoderm cells.

Methods, for selecting cells having one or more specific molecular markers are known in the art. For example, cells may be selected based on expression of various transcripts by flow cytometry, including fluorescence-activated cell sorting, or magnetic-activated cell sorting (see, e.g., International Patent Application No.: PCT/US2017/045496, the content of which is incorporated by reference herein in its entirety). In one embodiment, mesoderm cells are harvested after day 4 of differentiation and made into a single cell suspension. Cells are counted and prepared for antibody staining with anti-human antibodies to KDR, NCAM and APLNR. KDR⁺NCAM⁺APLNR⁺ cells are then gated/selected and sorted using flow cytometry. In certain embodiments, the sorting further comprises selection of SSEA5 KDR⁺NCAM⁺APLNR⁺ cells.

Activin A is a member of the TGF-β superfamily that is known to activate cell differentiation via multiple pathways. Activin A facilitates activation of mesodermal specification but is not critical for endothelial specification and subsequent endothelial cell proliferation. In one embodiment, the mesoderm differentiation medium comprises Activin A at a concentration of about 5-25 ng/ml. In a preferred embodiment, the endothelial differentiation medium comprises Activin A at a concentration of about 10 ng/ml.

Bone morphogenetic protein-4 (BMP-4) is a ventral mesoderm inducer that is expressed in adult human bone marrow (BM) and is involved in modulating proliferative and differentiative potential of hematopoietic progenitor cells (Bhardwaj et al. Nat Immunol. (2001) 2(2):172-80; Bhatia et al. J Exp Med. (1999) 189(7):1139-48; Chadwick et al. Blood. (2003) 102(3):906-15). Additionally, BMP-4 can modulate early hematopoietic cell development in human fetal, neonatal, and adult hematopoietic progenitor cells (Davidson and Zon, Curr Top Dev Biol. (2000) 50:45-60; Huber et al., Blood. (1998) 92(11):4128-37; Marshall et al., Blood. (2000); 96(4):1591-3). In one embodiment, the mesoderm differentiation medium comprises BMP-4 at a concentration of about 5-25 ng/ml. In one preferred embodiment, the endothelial differentiation medium comprises BMP-4 at a concentration of about 10 ng/ml.

Vascular endothelial growth factor (VEGF) is a signaling protein involved in embryonic circulatory system formation and angiogenesis. In vitro, VEGF can stimulate endothelial cell mitogenesis and cell migration. In one embodiment, the mesoderm differentiation medium comprises VEGF at a concentration of about 5-50 ng/ml. In a preferred embodiment, the endothelial differentiation medium comprises VEGF at a concentration of about 10 ng/ml.

Basic fibroblast growth factor, also referred to as bFGF or FGF-2, has been implicated in diverse biological processes, including limb and nervous system development, wound healing, and tumor growth. FGF-2 has been used to support feeder-independent growth of human embryonic stem cells. In one embodiment, the mesoderm differentiation medium comprises FGF-2 at a concentration of about 5-25 ng/ml. In a preferred embodiment, the endothelial differentiation medium comprises FGF-2 at a concentration of about 10 ng/ml.

In further embodiments, the isolated mesodermal precursor cells can be further induced to undergo endothelial differentiation in vitro according to methods well known in the art (e.g., see the published U.S. Patent Application No. 2017/0022476, the content of which is hereby incorporated herein in its entirety). For example, KDR⁺NCAM⁺APLNR⁺ mesoderm (MSD) precursor cells can be cultured in a chemically defined medium, e.g. Stemline II serum-free hematopoietic expansion medium, supplemented with an effective amount of the growth factors, VEGF, FGF-2 and BMP-4. After 10-12 days in culture, the MSD cells undergo endothelial differentiation. CD31⁺CD144⁺NRP-1⁺ ECFC-like cells can then be isolated using flow cytometry.

As used herein, the term “differentiation” refers to the developmental process of lineage commitment. A “lineage” refers to a pathway of cellular development, in which “precursor” or “progenitor” cells undergo progressive physiological changes to become a specified cell type having a characteristic function (e.g., endothelial cell). Differentiation occurs in stages, whereby cells gradually become more specified until they reach full maturity, which is also referred to as “terminal differentiation.” A “terminally differentiated cell” is a cell that has committed to a specific lineage and has reached the end stage of differentiation (i.e., a cell that has fully matured).

As used herein, “mesodermal differentiation medium” refers to any nutrient medium that supports and/or enhances differentiation of pluripotent cells into cells of the mesoderm lineage.

As used herein, “mesoderm” refers to the middle of three primary germ layers in an early embryo (the other two layers being ectoderm and endoderm). There are four components or classes of mesoderm, including axial mesoderm, paraxial mesoderm, intermediate mesoderm and lateral plate/extra-embryonic mesoderm. Mesoderm comprises “mesoderm cells”, also referred to as “mesodermal cells.”

As used herein, mesodermal (MSD) precursor cells refer to KDR⁺NCAM⁺APLNR⁺ cells. In certain embodiments, MSD cells refer to SSEA5⁻KDR⁺NCAM⁺APLNR⁺ cells. Under appropriate conditions disclosed herein, mesodermal (MSD) precursor cells can differentiate into ECFC-like cells and form blood vessels in vivo.

KDR (also known as CD309, “fetal liver kinase 1”, FLK1, “vascular endothelial growth factor receptor 2”, VEGFR or VEGFR2) refers to the Kinase Insert Domain Receptor (a Type III Receptor Tyrosine Kinase) or Vascular Endothelial Growth Factor Receptor 2 (EC:2.7.10.1), one of the two receptors for VEGF. KDR functions as the main mediator of VEGF-induced endothelial proliferation, survival, migration, tubular morphogenesis and sprouting. The signaling and trafficking of this receptor are regulated by multiple factors, including Rab GTPase, P2Y purine nucleotide receptor, integrin alphaVbeta3, T-cell protein tyrosine phosphatase.

NCAM (also known as Neural Cell Adhesion Molecule 1, the antigen recognized by monoclonal antibody 5.1H11, CD56 antigen, N-CAM-1, NCAM-1 or MSK39) refers to a cell adhesion protein which is a member of the immunoglobulin superfamily. The encoded protein is involved in cell-to-cell interactions as well as cell-matrix interactions during development and differentiation.

APLNR (also known as apelin receptor, HG11, angiotensin II receptor-like 1, angiotensin receptor-like 1, APJ receptor, AGTRL1 or APJR) refers to a member of the G protein-coupled receptor gene family. The encoded protein is related to the angiotensin receptor but is actually an apelin receptor that inhibits adenylate cyclase activity and plays a counter-regulatory role against the pressure action of angiotensin II by exerting hypertensive effect. It functions in the cardiovascular and central nervous systems, in glucose metabolism, in embryonic and tumor angiogenesis and as a human immunodeficiency virus (HIV-1) coreceptor.

SSEA5 refers to a monoclonal antibody (mAb) against hESCs, designated SSEA-5, which binds a novel antigen specifically expressed on hPSCs—the H type-1 glycan.

Differentiation of Mesodermal Precursor (MSD) Cells into Endothelial Progenitor Cells.

In certain embodiments, the present disclosure provides a method for generating an isolated population of engineered KDR⁺NCAM⁺APLNR⁺ mesodermal (MSD) precursor cells from engineered pluripotent stem cells. The method comprises providing engineered pluripotent stem cells (PSCs) as disclosed herein; inducing the pluripotent stem cells to undergo mesodermal differentiation, wherein the mesodermal induction comprises: i) culturing the pluripotent stem cells for about 24 hours in a mesoderm differentiation medium comprising Activin A, BMP-4, VEGF and FGF-2; and ii) replacing the medium of step i) with a mesoderm differentiation medium comprising BMP-4, VEGF and FGF-2 about every 24-48 hours thereafter for about 72 hours; and isolating from the cells induced to undergo mesoderm differentiation, wherein their isolation comprises: iii) sorting the cells to select for KDR⁺NCAM⁺APLNR⁺ mesoderm cells (see International Application No.: PCT/US2017/045496, the content of which is incorporated by reference herein in its entirety). In certain embodiments, the sorting further comprises selection of SSEA5 KDR⁺NCAM⁺APLNR⁺ cells.

In certain embodiments, the isolated mesodermal (MSD) precursor cells are further induced to undergo differentiation into endothelial progenitor cells, such as endothelial colony-forming-like cells. For example, KDR⁺NCAM⁺APLNR⁺ mesodermal (MSD) precursor cells can be cultured in a chemically defined medium, e.g. Stemline II serum-free hematopoietic expansion medium, supplemented with growth factors, e.g. VEGF, FGF-2 and BMP-4. After 10-12 days in culture, the MSD cells undergo endothelial differentiation. ECFC-like cells can then be isolated using flow cytometry.

As used herein, “endothelial colony-forming-like cells” or “ECFC-like cells” refer to non-primary endothelial progenitor cells that are generated in vitro from mesoderm (MSD) precursor cells. ECFC-like cells have various characteristics, at least including the potential to proliferate and form an endothelial colony from a single cell and have a capacity to form blood vessels in vivo in the absence of co-implanted or co-cultured cells. In certain embodiments, ECFC-like cells have properties similar to ECFCs isolated from blood, including (A) characteristic ECFC molecular phenotype; (B) capacity to form capillary-like networks in vitro on Matrigel™; (C) high proliferation potential; (D) self-replenishing potential; (E) capacity for blood vessel formation in vivo without co-culture with any other cells; (F) increased cell viability and/or decreased senescence and (G) cobblestone morphology. Importantly, as with ECFCs, the methods of generating ECFC-like cells described herein do not require co-culture with supportive cells, such as, for example, OP9 bone marrow stromal cells, embryoid body (EB) formation or exogenous TGF-β inhibition.

As used herein, “primary endothelial cells” refers to endothelial cells found in the blood, and which display a limited potential to proliferate and form an endothelial colony from a single cell and have a capacity to form blood vessels in vivo in the absence of co-implanted or co-cultured cells.

In certain embodiments, the ECFC-like cells express one or more markers chosen from CD31, NRP-1, CD144 and KDR. In one embodiment, the ECFC-like cells express two or more markers chosen from CD31, NRP-1, CD144 and KDR. In one embodiment, the ECFC-like cells express three or more markers chosen from CD31, NRP-1, CD144 and KDR. In one embodiment, the ECFC-like cells express four or more markers chosen from CD31, NRP-1, CD144 and KDR.

In certain embodiments, ECFC-like cells can have a high proliferation potential (HPP-ECFC-like). The terms “high proliferation potential”, “high proliferative potential” and “HPP” refer to the capacity of a single cell to divide into more than about 2000 cells in a 14-day cell culture. Preferably, HPP cells have a capacity to self-replenish. For example, the HPP-ECFC-like cells provided herein have a capacity to self-replenish, meaning that an HPP-ECFC-like cell can give rise to one or more HPP cells within a secondary HPP-ECFC colony when replated in vitro.

Various techniques for measuring proliferative potential of cells are known in the art and can be used with the methods provided herein to confirm the proliferative potential of the ECFC. For example, single cell assays such as those described in PCT publication WO 2015/138634 may be used to evaluate the clonogenic proliferative potential of ECFC. In general, an ECFC to be tested for proliferative potential may be treated to obtain a single cell suspension. The suspended cells are counted, diluted and single cells are cultured in each well of 96-well plates. After several days of culture, each well is examined to quantitate the number of cells. Those wells containing two or more cells are identified as positive for proliferation. Wells with ECFC counts of 1 are categorized as non-dividing, wells with ECFC counts of 2-50 are categorized as endothelial cell clusters (ECC), wells with ECFC counts of 51-500 or 501-2000 are categorized as low proliferative potential (LPP) cells and wells with ECFC counts of 2001 or greater are categorized as high proliferative potential (HPP) cells.

### Treatment of Perfusion Disorders

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with a perfusion disorder or disease, e.g. an ischemia-reperfusion (FR) injury. The term “treating” includes reducing or alleviating at least one adverse effect or symptom of a condition, disease or disorder associated with a perfusion disorder. Treatment is generally “effective” if one or more symptoms or clinical markers are reduced. Alternatively, treatment is “effective” if the progression of a perfusion disorder is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

“Prophylaxis” or “prophylactic” or “preventative” therapy as used herein includes preventing the condition from occurring or ameliorating the subsequent progression of the condition in a subject that may be predisposed to the condition but has not yet been diagnosed as having it.

The term “allogeneic,” as used herein, refers to cells of the same species that differ genetically to the cell in comparison.

The term “autologous,” as used herein, refers to cells derived from the same subject.

The term “engraft” as used herein refers to the process of stem cell incorporation into a tissue of interest in vivo through contact with existing cells of the tissue.

As used herein, the term “administering,” refers to the placement of a composition as disclosed herein into a subject by a method or route which results in at least partial delivery of the composition at a desired site. In certain embodiments, the disclosed compositions can be administered to an organ or tissue ex vivo followed transplantation into the patient.

In one embodiment, an “effective amount” refers to the optimal number of cells needed to elicit a clinically significant improvement in the symptoms and/or pathological state associated with a perfusion disorder including slowing, stopping or reversing cell death, reducing a neurological deficit or improving a neurological response. The therapeutically effective amount can vary depending upon the intended application or the subject and disease condition being treated, e.g., the weight and age of the subject, the severity of the disease condition, the manner of administration and the like, which can readily be determined by one of ordinary skill in the art, e.g., a board-certified physician.

Perfusion is the process by which a fluid passes through the circulatory system or lymphatic system of an organ, tissue, or extremity, e.g. the delivery of blood to a capillary bed in a tissue.

As used herein, a “perfusion disorder” or “perfusion disease” is any pathological process that deprives a subject's tissue, organ or extremity of oxygenated blood. A perfusion disorder can be caused by physical trauma or as a consequence of systemic or vascular disease that reduces arterial flow to an organ, tissue and/or extremity. Physical trauma can include, for example, a chronic obstructive process, or injury resulting from a physical insult such as frostbite or radiation.

As used herein, a “vascular disease” refers to a disease of the blood vessels, primarily arteries and veins, which transport blood to and from the heart, lungs, brain and peripheral organs such as, without limitation, the arms, legs, kidneys and liver. In particular, “vascular disease” refers to the coronary arterial and venous systems, the carotid arterial and venous systems, the aortic arterial and venous systems and the peripheral arterial and venous systems. The disease that may be treated is any that is amenable to treatment with the compositions disclosed herein, either as the sole treatment protocol or as an adjunct to other procedures such as surgical intervention. The disease may be, without limitation, atherosclerosis, vulnerable plaque, restenosis, peripheral arterial disease (PAD) or critical limb ischemia (CLI). Peripheral vascular disease includes arterial and venous diseases of the renal, iliac, femoral, popliteal, tibial and other vascular regions.

“Atherosclerosis” refers to the depositing of fatty substances, cholesterol, cellular waste products, calcium and fibrin on the inner lining or intima of an artery. Smooth muscle cell proliferation and lipid accumulation accompany the deposition process. In addition, inflammatory substances that tend to migrate to atherosclerotic regions of an artery are thought to exacerbate the condition. The result of the accumulation of substances on the intima is the formation of fibrous (atheromatous) plaques that can occlude the lumen of the artery, a process called stenosis. When the stenosis becomes severe enough, the blood supply to the organ supplied by the particular artery is depleted resulting in a stroke, if the afflicted artery is a carotid artery, or a heart attack if the artery is coronary, or loss of organ or limb function if the artery is peripheral.

Peripheral vascular diseases are generally caused by structural changes in blood vessels caused by such conditions as inflammation and tissue damage. A subset of peripheral vascular disease is peripheral artery disease (PAD). PAD is a condition that is similar to carotid and coronary artery disease in that it is caused by the buildup of fatty deposits on the lining or intima of the artery walls. Just as blockage of the carotid artery restricts blood flow to the brain and blockage of the coronary artery restricts blood flow to the heart, blockage of the peripheral arteries can lead to restricted blood flow to the kidneys, stomach, arms, legs and feet. In particular, a peripheral vascular disease can refer to a vascular disease of the superficial femoral artery.

“Critical limb ischemia” (CLI) is an advanced stage of peripheral artery disease (PAD). It is defined as a triad of ischemic rest pain, arterial insufficiency ulcers, and gangrene. The latter two conditions are jointly referred to as tissue loss, reflecting the development of surface damage to the limb tissue due to the most severe stage of ischemia. Over 500,000 patients in the U.S. each year are diagnosed with critical limb ischemia (CLI). Half the patients die from a cardiovascular cause within 5 years, a rate that is 5 times higher than a matched population without CLI (Varu et al. (2010) Journal of Vascular Surgery 51(1): 230-41; Rundback et al. Ann Vasc Surg. (2017) 38:191-205).

“Restenosis” refers to the re-narrowing of an artery at or near the site where angioplasty or another surgical procedure was previously performed to remove a stenosis. It is generally due to smooth muscle cell proliferation and, at times, is accompanied by thrombosis.

“Vulnerable plaque” refers to an atheromatous plaque that has the potential of causing a thrombotic event and is usually characterized by a thin fibrous cap separating a lipid filled atheroma from the lumen of an artery. The thinness of the cap renders the plaque susceptible to rupture. When the plaque ruptures, the inner core of usually lipid-rich plaque is exposed to blood. This releases tissue factor and lipid components with the potential of causing a potentially fatal thrombotic event through adhesion and activation of platelets and plasma proteins to components of the exposed plaque.

References and citations to other documents, such as patents, patent applications, patent publications, journals, books, papers, web contents, have been made in this disclosure. All such documents are hereby incorporated herein by reference in their entirety for all purposes. Any material, or portion thereof, that is said to be incorporated by reference herein, but which conflicts with existing definitions, statements, or other disclosure material explicitly set forth herein is only incorporated to the extent that no conflict arises between that incorporated material and the present disclosure material. In the event of a conflict, the conflict is to be resolved in favor of the present disclosure as the preferred disclosure.

### Examples

Examples have been set forth below for the purpose of illustration and to describe certain specific embodiments of the invention. However, the scope of the claims is not to be in any way limited by the examples set forth herein. Various changes and modifications to the disclosed embodiments will be apparent to those skilled in the art and such changes and modifications including, without limitation, those relating to the packaging vectors, cell lines and/or methods of the invention may be made without departing from the spirit of the invention and the scope of the appended claims.

The practice of the invention employs, unless otherwise indicated, conventional molecular biological and immunological techniques within the skill of the art. Such techniques are well known to the skilled worker and are explained fully in the literature. See, e.g., Bailey, J. E. and Ollis, D. F., Biochemical Engineering Fundamentals, McGraw-Hill Book Company, N Y, 1986; Current Protocols in Immunology, John Wiley & Sons, Inc., NY, N.Y. (1991-2015), including all supplements; Current Protocols in Molecular Biology, John Wiley & Sons, Inc., NY, N.Y. (1987-2015), including all supplements; Sambrook, et al., Molecular Cloning: A Laboratory Manual, 2nd Edition, Cold Spring Harbor, N.Y. (1989); and Harlow and Lane, Antibodies, a Laboratory Manual, Cold Spring Harbor, N.Y. (1989), all the contents of which are incorporated by reference herein in their entireties.

### Example 1: Reporter Tagging of the PSGL-1 Gene

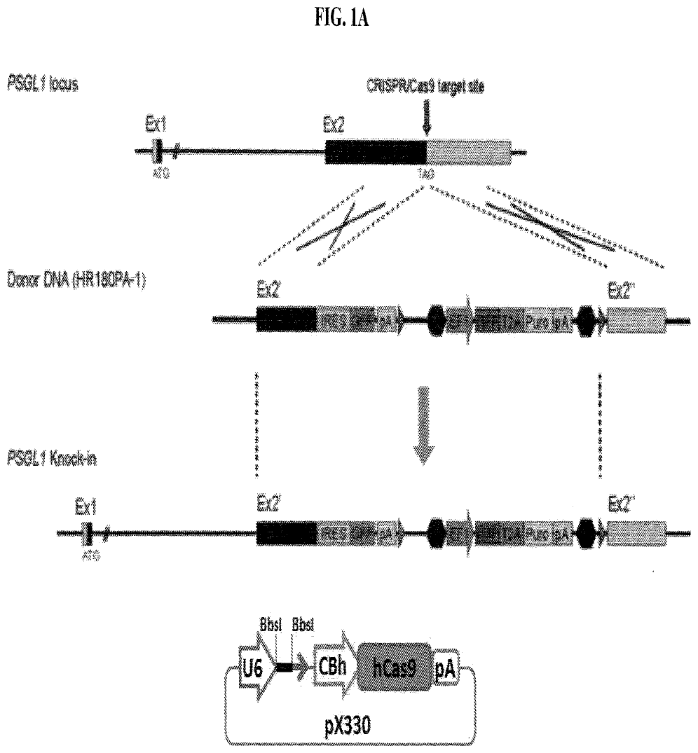

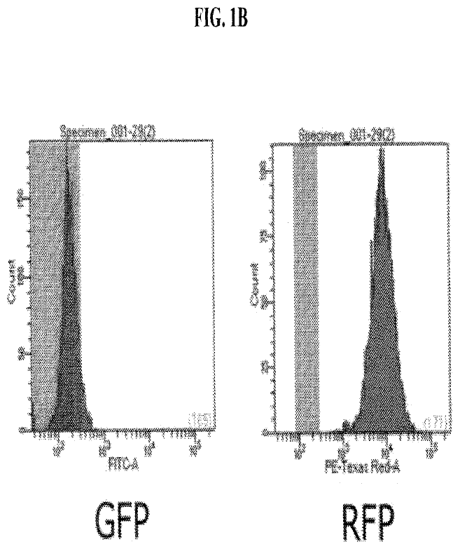

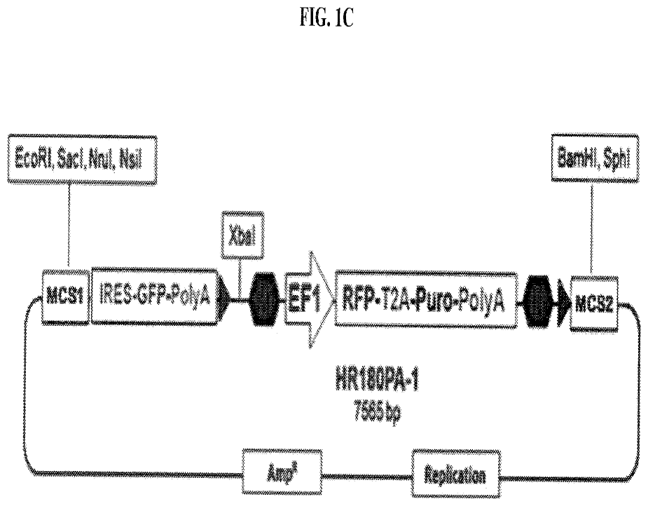

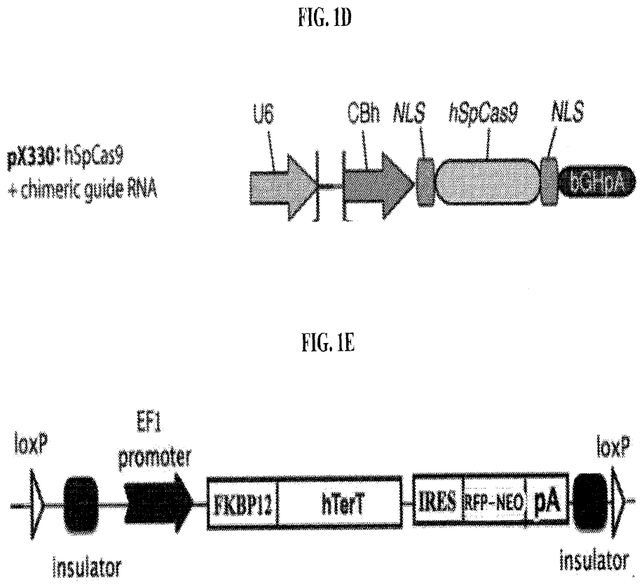

PSGL-1 is expressed concurrently with the appearance of mesodermal precursor cells in the iPSC cell population undergoing endothelial differentiation. Thus, in order to identify and isolate mesodermal precursor cells in the human iPSC population, CRISPR/Cas9 Homology-directed recombination was used to insert an IRES-GFP Co-Expression Homologous Recombination Targeting Vector HR180PA-1 into the 3′ untranslated region (UTR) of the endogenous PSGL-1 gene in human iPSCs (see FIG. 1A).

The HR180PA-1 vector comprising the IRES-GFP-pA-MCS1-EF1α-RFP-T2A-Puro-MCS2 cassette was purchased from System Biosystems (see FIG. 1C).

To knock IRES-GFP-pA-MCS1-EF1α-RFP-T2A-Puro-MCS2 into the 3′UTR of the endogenous PSGL-1 gene in the presence of CRISPR/Cas9, homologous PSGL-1 sequences immediately 5′ to the site of the targeted double strand break or DSB (5′ homology arm) were cloned by proofreading PCR into MCS1 upstream of the IRES-GFP-pA of HR180PA-1. Similarly, homologous PSGL-1 sequences immediately 3′ to the DSB (3′ homology arm) were amplified by proofreading PCR and cloned into the MCS2 site downstream of the EF1α-RFP-T2A-Puro cassette (see FIG. 1A). Standard PCR and cloning procedures are described in detail in the PrecisionX™ HR Targeting Vectors User Manual (System Biosystems).

To ensure the expression from the EF1α promoter in iPSCs was not inactivated by epigenetic modifications or influenced by neighboring sequences, the EF1α-RFP-T2A-Puro cassette was flanked by insulator sequences (denoted by a black hexagonal symbol in FIG. 1A). The EF1α promoter drives the expression of a bicistronic mRNA encoding red fluorescent protein (RFP) and a puromycin resistance gene (PURO) separated by a 2A self-cleaving peptide (T2A; (GSG) E G R G S L L T C G D V E E N P G P (SEQ ID NO: 21)). T2A peptides are short peptides (about 20 amino acids) that allow for the translation of equimolar levels of multiple genes from the same mRNA. The EF1α-RFP-T2A-Puro cassette is also flanked by LoxP sites. Thus, the EF1α-RFP-T2A-Puro cassette can be excised by expression of Cre recombinase, leaving only the PSGL-1 gene-IRES-eGFP construct and a single LoxP site. Removal of the EF1α-RFP-T2A-Puro cassette allows the knock-in of the PSGL1-HR180PA-1 vector into the other PSGL-1 allele.

The pX330 plasmid was obtained from Addgene (plasmid #42230; first described by Cong et al. Science (2013) 339(6121):819-23). The pX330 expression cassette comprises a CBh promoter (a ˜800 bp hybrid promoter between the immediate-early cytomegalovirus (CMV) and the chicken β-actin (CBA) promoters) driving the expression of a human codon-optimized SpCas9 and a U6 promoter driving the expression of a chimeric guide RNA targeting the 3′UTR of the PSGL-1 gene (seeFIG. 1D). The DSB cleavage site is engineered to be 2-3 bp upstream of the protospacer adaptor motif (PAM) immediately following the guide RNA sequence.

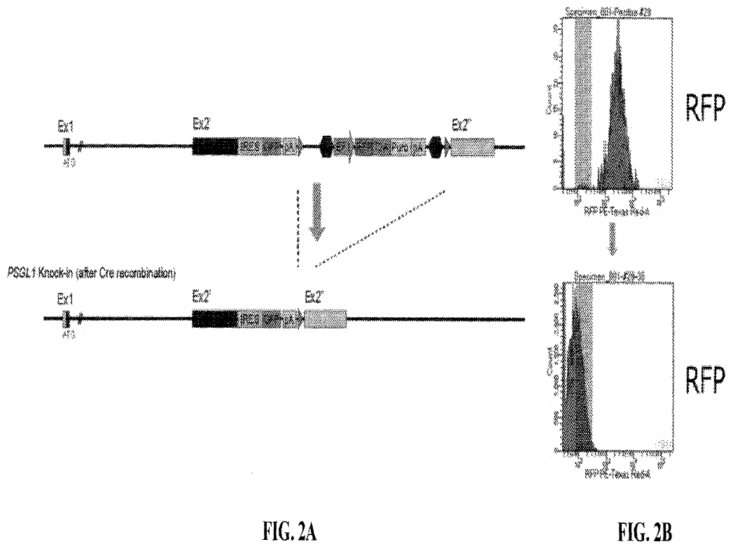

The PSGL1-HR180PA-1 and pX330 plasmids were transfected into iPSCs by electroporation according to standard procedure. Stably transfected colonies were then screened for puromycin resistance and red fluorescence (see FIG. 1B). The EF1α-RFP-T2A-Puro cassette was then excised by expression of Cre recombinase (see FIG. 2A), leaving only the PSGL1-IRES-eGFP construct and a single LoxP site. Cre recombination between the loxP sites resulted in the extinction of red fluorescence (see FIG. 2B).

### Example 2: Mesodermal Differentiation of PSGL1-IRES-GFP iPSCs

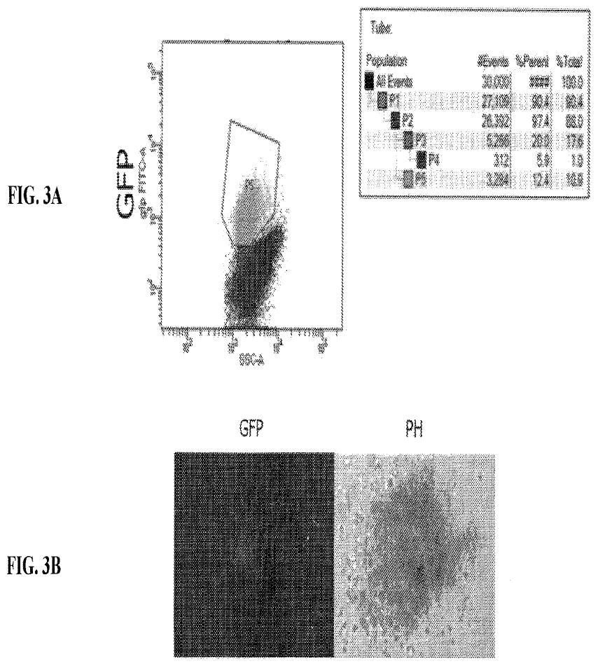

PSGL1-IRES-eGFP iPSCs were cultured for 2 days (−D2) in mTeSR1 media. Cultures were then directed toward the mesodermal lineage by the addition of activin A (10 ng/mL) in the presence of FGF-2, VEGF165, and BMP4 (10 ng/mL) for 24 hrs. The following day (D1), activin-A containing media was removed and replaced with 8 mL of Stemline II complete media (Sigma) containing FGF-2 (Stemgent), VEGF165 (R&D) and BMP4 (R&D). Media was replaced with 8 ml of fresh Stemline II differentiation media on day 3. On day 4, the cells were sorted by FACS for green fluorescence (see FIG. 3A).

FIG. 3B shows the appearance of green fluorescence in cells containing the PSGL1 Gene Knock-in after differentiation of iPSCs into colony-forming unit-granulocyte, erythroid, macrophage, megakaryocyte (CFU-GEMM) colonies. CFU-GEMM colonies include myeloid cells that are known to express PSGL1 (Laszik et al., Blood, (1996) 88, No. 8, 3010-3021).

### Example 3: Dual Reporter Tagging of Both Alleles of the Safe Harbor CLYBL Gene

Transcription activator-like effector nucleases (TALENs) were used to knock-in simultaneously a cGFP or td tomato reporter gene into one of the CLYBL alleles in human induced pluripotent stem cells (iPSCs) as described previously by Cerbini et al. (2015) PLoS One 10(1):e0116032.

Human iPSCs were passaged two days before transfection using StemPro Accustase at an ap-propriate density to achieve roughly 80% confluency in 48 hours. For transfection, 3×10⁶ cells were harvested with Accutase. Cells were resuspended in 100 ml P3 Primary Cell 4D-Nucleofector X Solution (Lonza # V4XP-3024) with 5 mg each TALEN and 10 mg pC13N-iCAG.tdTomato and/or pC13N-iCAG.cGFP donor plasmid and transfected using the 4D-Nucleofector X Unit (Lonza # AAF-1001X) and preset program CB-150. Cells were replated in 3 wells of a 6-well plate and stable transfectants were selected by drug selection (puromycin and neomycin). TALEN vectors and the pC13N-iCAG.tdTomato and pC13N-iCAG.cGFP vector were purchased from Addgene.

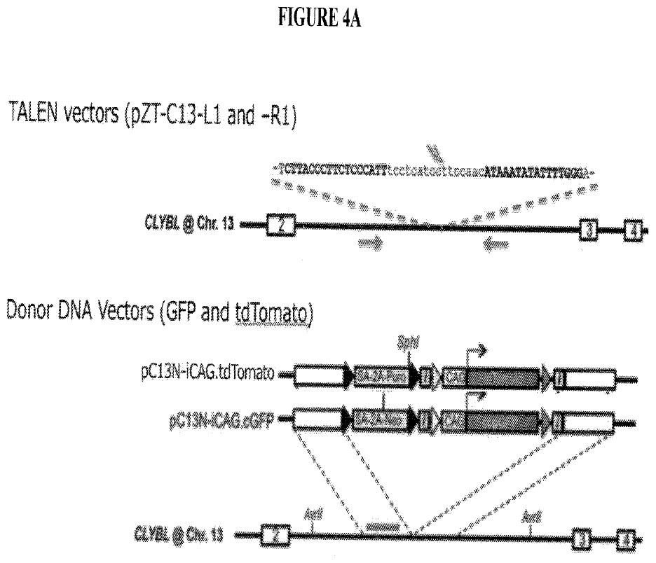

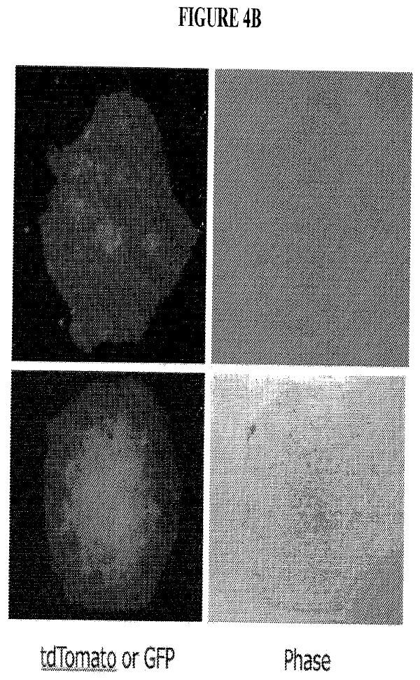

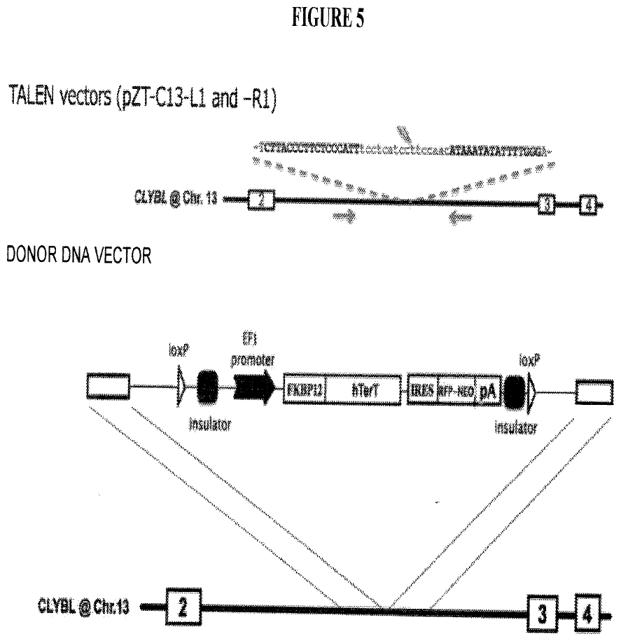

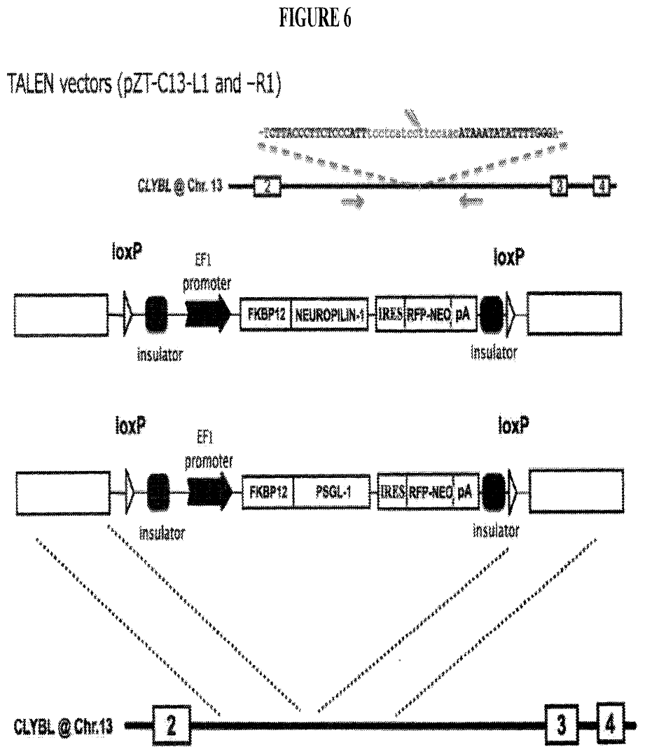

FIG. 4B shows GFP/tdTomato expression in iPSC colonies having the GFP/tdTomato reporter transgenes knocked-in to both alleles of the endogenous CLYBL locus.

### Prophetic Example 4: Generation of Transiently Immortalized Mesodermal Precursor Cells

Mesodermal (MSD) precursor cells proliferate in culture for only 1-3 days before undergoing terminal differentiation and/or replicative senescence. To sustain cell proliferation, MSD cells are engineered to express one or more immortalizing genes such as telomerase.

In this Example, transcription activator-like effector nucleases (TALENs) are used to knock-in an inducible hTERT expression donor vector into one of the CLYBL alleles of PSGL1-IRES-GFP iPSCs (see FIG. 5) as described above.

The donor vector is engineered as shown in FIG. 1E.

A 12-kDa ligand-dependent destabilization domain (MGVQVETISP (SEQ ID NO: 22), derived from an unstable FKBP12 mutant, is fused in frame to the N-terminus of human telomerase having the amino acid sequence of SEQ ID NO: 5 and cloned between an EF1a promoter and an IRES-RFP-Neo module (commercially available from Biosettia as a lentiviral vector, pLV-EF1a-MCS-IRES-RFP-Neo). The EF1a-FKBP12-hTerT-IRES-RFP-Neo construct is also flanked by insulator elements and by loxP sites. Methods and compositions for the rapid and reversible destabilizing of specific proteins using cell-permeable, synthetic molecules was previously in U.S. Pat. No. 8,173,792, the content of which is incorporated by reference herein in its entirety).

PSGL1-IRES-GFP iPSCs are maintained on hESC-qualified Matrigel Basement Membrane Matrix (BD #354277) and cultured with Essential 8 Medium (Invitrogen # A14666SA) as per each manufacturer's instructions. Media is refreshed daily. For passaging, dissociation buffer is made by adding 500 ml 0.5M EDTA and 0.9 g NaCl into 500 ml of Calcium and Magnesium free PBS (Invitrogen #14190). Cells are routinely passaged at 80% confluence.

The PSGL1-IRES-GFP iPSCs are passaged two days before transfection using StemPro Accustase at an appropriate density to achieve roughly 80% confluency in 48 hours. For transfection, 3×10⁶ cells are harvested with Accutase. Cells are resuspended in 100 ml P3 Primary Cell 4D-Nucleofector X Solution (Lonza # V4XP-3024) with 5 mg each TALEN and 10 mg of the EF1a-FKBP12-hTerT-IRES-RFP-Neo donor plasmid and transfected using the 4D-Nucleofector X Unit (Lonza # AAF-1001X) and preset program CB-150. Cells are replated onto DR4 MEFs (GlobalStem # GSC-6004G-C) in 3 wells of a 6-well plate and E8 media is supplemented with 10 mM ROCK inhibitor Y27632 for 24 hours post-nucleofection.

G418 concentration is first optimized on untargeted PSGL1-IRES-GFP iPSCs by a kill-curve analysis. At 2-3 days post nucleofection, NutriStem XF/FF medium (Stemgent #01-0005) supplemented with 25 mg/ml G418 is used to replace E8 medium and refreshed every day for up to 7-12 days or until selection appears complete (i.e., when untargeted control cells are all killed). All drug-resistant clones are then picked and expanded in E8/Matrigel culture condition.

The transformed PSGL1-IRES-GFP iPSCs having the FKBP12-telomerase transgene expression cassette integrated into one of the alleles of the CLYBL locus are cultured for 2 days (−D2) in mTeSR1 media. Cultures are then differentiated toward the mesodermal lineage by the addition of activin A (10 ng/mL) in the presence of FGF-2, VEGF165, and BMP4 (10 ng/mL) for 24 hrs. The following day (D1), activin-A containing media is removed and replaced with 8 mL of Stemline II complete media (Sigma) containing FGF-2 (Stemgent), VEGF165 (R&D) and BMP4 (R&D). Media is replaced with 8 ml of fresh Stemline II differentiation media on day 3.

On day 4, the appearance of mesodermal (MSD) precursor cells is detected by the activation of PSGL1 induced green fluorescence. The small-molecule rapamycin analogue Shield 1 is added to the culture media (commercially available through Takara Bio). Addition of the Shield 1 ligand restores the expression of telomerase protein in a dose-dependent manner. The GFP positive mesodermal precursor cells are then be cultured in vitro. GFP positive KDR⁺NCAM⁺APLNR⁺ mesodermal precursor cells are selected by FACS analysis. After expansion of the mesodermal precursor cells, the EF1a-FKBP12-HTert-IRES-RFP-Neo expression cassette can be excised by protein transduction with Tat-Cre recombinase leaving behind a single LoxP site at the CLYBL locus. The excision event can be monitored by the extinction of red fluorescence.

To maintain the proliferation of the mesodermal precursor cells in the absence of the EF1a-FKBP12-HTert-IRES-RFP-Neo expression cassette, Tat-telomerase fusion protein can be added to the culture media as needed.

### Prophetic Example 5: Isolating Non-Transgenic Mesodermal (MSD) Precursor Cells

PSGL1-IRES-GFP iPSCs are added to a population of iPSC cells in a ratio of 1:100 to 1:10000. The cells are cultured for 2 days (−D2) in mTeSR1 media before being differentiated toward the mesodermal lineage by the addition of activin A (10 ng/mL) in the presence of FGF-2, VEGF165, and BMP4 (10 ng/mL) for 24 hrs. The following day (D1), activin-A containing media is removed and replaced with 8 mL of Stemline II complete media (Sigma) containing FGF-2 (Stemgent), VEGF165 (R&D) and BMP4 (R&D). Media is replaced with 8 ml of fresh Stemline II differentiation media on day 3. On day 4, the appearance of mesodermal (MSD) precursor cells is detected by the activation of PSGL1 induced green fluorescence at which point VP22-hTERT is added to the culture media to maintain cellular proliferation. Methods of administering a transducible protein to cultured cells are described in e.g. WO2000034308 and WO2002055684, the contents of which are incorporated by reference herein in their entireties. Following expansion of the cell population, PSGL1-IRES-GFP mesodermal MSD precursor cells are removed by FACS leaving a population non-transgenic mesodermal MSD precursor cells.

### Prophetic Example 6: Overexpression of PSGL1 and/or Neuropilin-1 in Mesodermal Precursor Cells

To overexpress PSGL1 or Neuropilin-1 in mesodermal (MSD) precursor cells, the PSGL1 (SEQ ID NO: 2) or the Neuropilin gene (SEQ ID NO: 4) is cloned into the multiple cloning site (MCS) of the EF1a-MCS-IRES-RFP-Neo donor vector. The PSGL1 or Neuropilin-1 donor vectors are then (nucleo-) transfected with CLYBL TALEN vectors into human PSGL1-IRES-GFP iPSCs as described in Example 4.

At 2-3 days post nucleofection, NutriStem XF/FF medium (Stemgent #01-0005) supplemented with 25 mg/ml G418 are used to replace E8 medium and refreshed every day for up to 7-12 days or until selection appeared complete (when untargeted control cells are all killed). All drug-resistant red fluorescent clones are picked and expanded in E8/Matrigel culture condition.

The transformed PSGL1-IRES-GFP iPSCs having the FKBP12-PSGL-1 or FKBP12-Neuropilin-1 transgene expression cassette integrated into one of the alleles of the CLYBL locus are cultured for 2 days (−D2) in mTeSR1 media. Cultures are then differentiated toward the mesodermal lineage by the addition of activin A (10 ng/mL) in the presence of FGF-2, VEGF165, and BMP4 (10 ng/mL) for 24 hrs. The following day (D1), activin-A containing media is removed and replaced with 8 mL of Stemline II complete media (Sigma) containing FGF-2 (Stemgent), VEGF165 (R&D) and BMP4 (R&D). Media is replaced with 8 ml of fresh Stemline II differentiation media on day 3.

On day 4, the appearance of mesodermal (MSD) precursor cells is detected by the activation of PSGL1 induced green fluorescence. To maintain proliferation of the MSD cells, a recombinant PTD-telomerase protein (e.g. Tat-telomerase) is added to the culture media. Following cell proliferation in vitro, the cells are tested for the concurrent expression of PSGL-1 induced GFP fluorescence and the markers KDR, NCAM and APLNR using FACS analysis. Aliquots of the cells are then collected and frozen for storage using standard procedures.

### Prophetic Example 7: Enhanced Migration of Mesodermal Precursor Cells to Sites of Ischemic Injury

Transgenic FKBP12-fusion protein (i.e., either PSGL-1 or Neuropilin-1) mesodermal (MSD) precursor cells (see Example 6) are cultured for 3-4 days in mTeSR1 media supplemented with Tat-telomerase. Prior to administration to an animal model of peripheral artery disease, the small-molecule rapamycin analogue Shield 1 (commercially available through Takara Bio) or PBS (control) is added to the media for 12-24 hours. Addition of the Shield 1 ligand enhances the stability of the FKBP12-fusion protein in a dose-dependent manner.

The ability of transgenic MSD cells to migrate to sites of ischemic injury is then tested in a previously described animal model of hindlimb ischemia. Briefly, the left femoral artery of anesthetized 12-week-old Balb/C or “nude” mice weighing between 26 g and 30 g is exposed, dissected free, and excised (see Kalka et al., Proc. Natl Acad Sci USA. (2000) 28; 97(7):3422-7; Madeddu et al. FASEB J. (2004)18(14):1737-9). One day after operative excision of one femoral artery, mice receive an intramuscular injection of 1-2000×10³ cells treated with either Shield1 ligand or PBS. 7-14 days after treatment, the mice are sacrificed, and the site of ischemia is tested for the presence of red fluorescent cells indicating the presence of cells derived from the transgenic MSD cells. Expression of stabilized PSGL-1 or Neuropilin enhances the migration of the transgenic MSD cells to the site of ischemia as compared to transgenic MSD cells treated with PBS.

1. An isolated population of engineered mesodermal precursor cells expressing at least one of KDR, NCAM and APLNR, wherein the precursor cells are engineered to enhance the non-neoplastic proliferation and survival of the mesodermal precursor cells and/or the migration of the mesodermal precursor cells and their progeny toward ischemic tissue.

2. The population of engineered mesodermal precursor cells of claim 1, wherein the mesodermal precursor cells express at least two of KDR, NCAM and APLNR.

3. The population of engineered mesodermal precursor cells of claim 1, wherein the mesodermal precursor cells express KDR, NCAM and APLNR.

4. The population of engineered mesodermal precursor cells of any claim 1, wherein the mesodermal precursor cells can differentiate into endothelial progenitor cells.

5. The population of engineered mesodermal precursor cells of any claim 4, wherein the endothelial progenitor cells comprise endothelial colony forming-like cells (ECFC-like).

6. The population of engineered mesodermal precursor cells of claim 1, wherein the mesodermal precursor cells are engineered by gene editing.

7. The population of engineered mesodermal precursor cells of claim 1, wherein the mesodermal precursor cells comprise an agent, wherein the agent enhances the non-neoplastic proliferation and survival of the mesodermal precursor cells and/or the migration of the mesodermal precursor cells and their progeny toward ischemic tissue.

8. The population of engineered mesodermal precursor cells of claim 7, wherein the agent comprises a transgene and/or an mRNA.

9. The population of engineered mesodermal precursor cells of claim 8, wherein the transgene or mRNA encoding P selectin ligand 1 (PSGL-1) comprises a nucleotide sequence having at least 25 nucleotides of SEQ ID NO: 2, and wherein the expression of the transgene or mRNA enhances the migration of the mesodermal precursor cells and their progeny toward ischemic tissue in vivo.

10. The population of engineered mesodermal precursor cells of claim 8, wherein the transgene or mRNA encoding neuropilin-1 comprises a nucleotide sequence having at least 25 nucleotides of SEQ ID NO: 4 and wherein the expression of the transgene or mRNA enhances the migration of the mesodermal precursor cells and their progeny toward ischemic tissue in vivo.

11. The population of engineered mesodermal precursor cells of claim 8, wherein the transgene or mRNA encoding telomerase comprises a nucleotide sequence having at least 25 nucleotides of SEQ ID NO: 6, wherein the expression of the transgene or mRNA enhances the non-neoplastic proliferation and survival of the mesodermal precursor cells.

12. The population of engineered mesodermal precursor cells of claim 7, wherein the agent comprises a transducible protein.

13. The population of engineered mesodermal precursor cells of claim 12, wherein the transducible protein comprises P selectin ligand 1 (PSGL-1), neuropilin-1 and/or telomerase or any portion thereof.

14. The population of engineered mesodermal precursor cells of claim 8, wherein the expression of the transgene is inducible.

15. The population of engineered mesodermal precursor cells of claim 8, wherein the transgene is episomal.

16. The population of engineered mesodermal precursor cells of claim 8, wherein the transgene is chromosomally integrated.

17. The population of engineered mesodermal precursor cells of claim 8, wherein the transgene is inserted into a genomic safe harbor site.

18. The population of engineered mesodermal precursor cells of claim 8, wherein the transgene is operably linked to a promoter of an endogenous gene that is expressed in the mesodermal precursor cell population.

19. The population of engineered mesodermal precursor cells of claim 8, wherein the transgene is placed downstream of an internal ribosomal entry site (IRES) and inserted into the 3′ untranslated region of an endogenous gene that is expressed in the mesodermal precursor cell population.

20. The population of engineered mesodermal precursor cells of claim 18, wherein the endogenous gene has a nucleotide sequence comprising at least 25 nucleotides of SEQ ID NO: 2 or SEQ ID NO: 4.

21. The population of engineered mesodermal precursor cells of claim 1, wherein the mesodermal precursor cells are derived from pluripotent stem cells expressing at least one stem cell transcription factor selected from the group consisting of NANOG, SOX2 and OCT4A.

22. The population of engineered mesodermal precursor cells of claim 21, wherein the pluripotent stem cells are induced pluripotent stem cells (iPSCs).

23. A cell composition comprising a first cell population of engineered mesodermal precursor cells of claim 1 and a second non-recombinant cell population.

24. The cell composition of claim 23, wherein the second non-recombinant cell population comprises mesodermal precursor cells.

25. A method for treating a perfusion disorder in a subject's organ, tissue and/or extremity comprising administering a cellular composition comprising a therapeutically effective amount of the transgenic mesodermal precursor cells of any one of the preceding claims.

26. The method of claim 25, wherein the subject's organ, tissue and/or extremity is irradiated prior to the administration of the cellular composition.

27. The method of claim 25, wherein the subject's perfusion disorder is caused by physical trauma to the subject's organ, tissue and/or extremity.

28. The method of claim 25, wherein the subject's perfusion disorder is a vascular disorder.

29. The method of claim 28, wherein the vascular disorder causes an ischemia and/or reperfusion injury to the subject's organ, tissue and/or extremity.

30. The method of claim 28, wherein the vascular disorder is peripheral arterial disease (PAD) or critical limb ischemia (CLI).

31. The method of claim 25, wherein the subject's organ or tissue is from the musculoskeletal system, circulatory system, nervous system, integumentary system, digestive system, respiratory system, immune system, urinary system, reproductive system or endocrine system.

32. The method of claim 25, wherein the organ is the subject's heart, lung, brain, liver and/or kidney.

33. The method of claim 25, wherein the tissue is an epithelial, connective, muscular, and/or nervous tissue.

34. The method of claim 25, wherein the tissue is cerebral, myocardial, lung, renal, liver, skeletal, and/or peripheral tissue.

35. The method of claim 25, wherein the administration of the cellular composition enhances blood flow through the subject's organ, tissue and/or extremity.

36. The method of claim 25, wherein the administration of the cellular composition restores endothelial cell function in the subject's organ, tissue and/or extremity.

37. The method of claim 25, wherein the administration of the cellular composition promotes neovascularization in the subject's organ, tissue and/or extremity.

38. The method of claim 25, wherein the cellular composition is administered directly to the subject's organ, tissue and/or extremity in vivo.

39. The method of claim 25, wherein the cellular composition is administered directly to the subject's organ and/or tissue ex vivo.

40. The method of claim 39, wherein, after the administration, the organ and/or tissue is transplanted into the subject.

41. The method of claim 25, wherein the cellular composition is administered intravenously to the subject.

42. The method of claim 25, wherein the subject has atherosclerosis, diabetes and/or cancer.
